(19) United States
(12) Patent Application Publication
(10) Pub. No.: US 2024/0015445 A1
(43) Pub. Date: Jan. 11, 2024

(54) NON-CONTACT SENSOR SYSTEMS AND METHODS

(71) Applicant: LEVEL 42 AI INC., Mountain View, CA (US)

(72) Inventors: Andreas SCHUH, Mountain View, CA (US); Michael MORIMOTO, Mountain View, CA (US); Nelson L. JUMBE, Mountain View, CA (US); Mark FREEMAN, Mountain View, CA (US)

(21) Appl. No.: 18/043,981

(22) PCT Filed: Sep. 3, 2021
(86) PCT No.: PCT/US2021/049162

§ 371 Date: Mar. 3, 2023

(30) Foreign Application Priority Data
May 8, 2021 (WO) PCT/IB2021/053919

(51) Int. Cl. H04R 9/08; A61B 7/04; A61B 5/00

(52) U.S. Cl. CPC H04R 9/08; A61B 7/04; A61B 5/7225; A61B 5/7267; A61B 2562/0204; A61B 2503/40

(57) ABSTRACT

A system for non-contact monitoring of acoustic signals associated with a body, the system comprising: a sensing device comprising: a support member defining an aperture, a diaphragm extending across the aperture such that at least a portion of the diaphragm covers the aperture, and a sensor connected to the support member or the membrane and configured to convert movement of the diaphragm to electric signal data.

### CROSS-REFERENCE

This application claims the benefit of U.S. Provisional Patent Application Ser. No. 63/075,056 filed Sep. 4, 2020; U.S. patent application Ser. No. 17/096,806 filed Nov. 12, 2020, PCT/IB2021/053919 filed May 8, 2021; and PCT/US2021/046566 filed Aug. 18, 2021. The contents of the aforementioned applications are incorporated by reference herein in their entirety. The content of co-pending PCT application entitled Secure Identification methods and systems filed on Sep. 3, 2021, is also incorporated by reference in its entirety.

### TECHNICAL FIELD

This invention relates generally to the field of non-contact sensor systems for monitoring of signals associated with a body, such as but not exclusively, vibroacoustic signals associated with a human subject for monitoring a condition of the subject.

### BACKGROUND

For monitoring of a condition of a body such as a human or animal subject, traditionally, medical care practitioners utilize a suite of instruments, each specialized to detect a particular biometric of the subject. However, a comprehensive assessment of the subject typically requires an array of different instruments. This presents certain challenges such as greater complexity, steeper learning curves for proper use, greater cost, and relative lack of portability and data interoperability.

Furthermore, many conventional instruments require contact with the skin or clothing of the subject. One such instrument is a stethoscope which is used to detect audible body sounds of a patient, such as those generated by the heart, lungs, and gastrointestinal systems of the subject. However, such contact-based instruments and their associated methods of use are not feasible for mass screening of many bodies, nor for rapid monitoring or testing of a given condition, such as a viral infection. Furthermore, sounds within the audible range may be of limited use.

Accordingly, there is a need for sensor systems that overcome or minimize the above-mentioned problems.

### SUMMARY

Embodiments of the present disclosure reduce or overcome the disadvantages of the aforementioned conventional sensor systems.

Broadly, Developers have discovered that vibroacoustic signals associated with a body and having frequencies that extend beyond the audible range can be used to detect and/or monitor certain conditions associated with the body. More specifically, acoustic signals having an overall bandwidth ranging from about 0.01 Hz to at least about 50 kHz, from about 0.01 Hz to at least about 60 kHz, from about 0.01 Hz to at least about 70 kHz, from about 0.01 Hz to at least about 80 kHz, from about 0.01 Hz to at least about 90 kHz, from about 0.01 Hz to at least about 100 kHz, from about 0.01 Hz to at least about 110 kHz, from about 0.01 Hz to at least about 120 kHz, from about 0.01 Hz to at least about 130 kHz, from about 0.01 Hz to at least about 140 kHz, from about 0.01 Hz to at least about 150 kHz, from about 0.01 Hz to at least about 160 kHz, from about 0.01 Hz to more than about 150 kHz.

Developers have developed sensor systems and methods that can detect, in a non-contact manner, such vibroacoustic signals with a frequency range including the audible range and extending beyond the audible range. Neither direct contact (e.g. skin contact) nor indirect contact (e.g. through clothing or fur) with the body being monitored is required. Advantageously, the sensor systems and methods of the present disclosure are non-invasive.

In certain embodiments, the sensor systems and methods of the present disclosure can operate with sensor components spaced, such as by air, at a distance of about 1 mm, 2 mm, 5 mm, 1 cm, 5 cm, 10 cm, 1 meter, 2 meter, 3 meter, 4 meter, 5 meter, 6 meter, 7 meter, 8 meter, 9 meter or 10-50 meters from the body.

In certain embodiments, sensor systems and methods of the present disclosure may be well suited for detecting infectious bodily conditions, such as viral infections, e.g. Covid-19. Current Covid-19 screening approaches are either simple and fast but lack accuracy (e.g., temperature checks), or are accurate but neither simple nor fast (e.g., antibody screening). Current screening approaches, therefore, are impractical, inconvenient, cannot mass-screen, present a delay between testing and the results, and do not identify individuals at early infection stages. Unlike current screening approaches, embodiments of the present technology can monitor a number of bodies at the same time, and determine in real-time for each body whether there is a Covid-19 infection.

Generally, in some embodiments, the present systems comprise a sensor platform. The sensor platform may include a sensing device such as a vibroacoustic sensor including one or more sensors configured to detect a vibroacoustic signal, a signal processing system configured to extract, from the detected vibroacoustic signal, a vibroacoustic signal component originating from a subject, and at least one processor configured to characterize a bodily condition of the subject based at least in part on the extracted vibroacoustic signal component using, for example, a machine learning model. In some variations, the bodily condition of a subject may include a health condition of a living subject or a physical characterization of a non-living subject.

From another aspect, there is provided a system for non-contact monitoring of a body, the sensing system comprising: a sensing device having a frame, a sensor for detecting vibroacoustic signals connected to the frame, and a diaphragm extending across at least a portion of the frame and connected thereto, the diaphragm also being connected to at least a portion of the sensor.

From another aspect, there is provided a system for non-contact monitoring of acoustic signals associated with a body, the system comprising: a sensing device comprising: a support member defining an aperture, a diaphragm extending across the aperture such that at least a portion of the diaphragm covers the aperture, and a sensor connected to the support member or the membrane and configured to convert movement of the diaphragm to electric signal data.

In certain embodiments, the sensor is configured to detect acoustic signals having a frequency ranging from about 0.01 Hz to at least about 160 kHz.

In certain embodiments, the system further comprises a computing system, including a processor, communicatively coupled to the sensing device and configured to execute a method for determining a bodily condition of the body based on the electric signal data.

In certain embodiments, the processor is configured to filter the electric signal data to remove electric data not associated with the body, the determining the bodily condition being based on the filtered electric signal data.

In certain embodiments, the body is a human or animal subject, and the filtering the electric signal data comprises the processor removing electric signal data which is not associated with a physiological parameter of the human or animal subject.

In certain embodiments, the method for determining a bodily condition based on the electric signal comprises executing a trained machine learning algorithm.

In certain embodiments, the support member is a frame having a first side and a second side and the aperture extends through the frame between the first side and the second side, wherein the diaphragm covers the aperture on one of the first side and the second side.

In certain embodiments, the sensing device further comprises a back cover to cover the aperture on the other of the first side and the second side.

In certain embodiments, the diaphragm is configured to seal the aperture. The seal may be a fluid seal or an acoustic seal.

In certain embodiments, the support member comprises a frame having a first side and a second side, wherein the aperture is formed in one of the first side and the second side and does not extend therethrough.

In certain embodiments, the sensor comprises: a voice coil component comprising a coil holder supporting wire windings; a magnet component comprising a magnet supported by a magnet housing, the magnet having a magnet gap configured to receive at least a portion of the voice coil component in a spaced and moveable manner; a connector connecting the voice coil component to the magnet component, the connector being compliant and permitting relative movement of the voice coil component; wherein one of the voice coil component and the magnet component is connected to the diaphragm such that movement of the diaphragm induces a relative movement between the voice coil component and the magnet component.

In certain embodiments, the diaphragm is attached to the voice coil component and the wire windings are spaced from the diaphragm.

In certain embodiments, wherein the sensor comprises an electric potential sensor which is attached to the support member and spaced from the diaphragm. The electric potential sensor may comprise an electrode layer, a guard layer, a GND layer and a circuit layer. The diaphragm may include a layer of a conductive material.

In certain embodiments, the electric potential sensor is positioned in a cavity of the aperture, or outside of the cavity.

In certain embodiments, the sensor is one or more selected from: a voice-coil type sensor, an electric potential sensor, a capacitive sensor, a magnetic field disturbance sensor, a photodetector and light source, a strain sensor, an Inertial Measurement Unit (IMU), and an acoustic echo doppler.

In certain embodiments, the system further comprises a plurality of sensors arranged as an array relative to the support member. Each sensor may be supported by a respective support member. An outer mount may be provided to which the support members are attached. The plurality of support members may be planar. The diaphragm may be common to the plurality of sensors and support members. In other words, the diaphragm may cover the respective apertures of all of the support members. The diaphragm may be attached to each support member around a periphery thereof to close or fluidly seal the respective aperture. Alternatively, the diaphragm may be attached to the outer mount to close or fluidly seal the aperture of each of the support members therein.

Each sensor of the plurality of sensors may be supported by a sub-frame of the support member. The diaphragm may be connected to each sub-frame. The diaphragm may fluidly seal about each sub-frame. At least two of the sub-frames may be spaced from one another.

In certain embodiments, each sensor of the plurality of sensors is configured to detect a different frequency range of acoustic signals.

In certain embodiments, the sensing device further comprises a front cover connected to the support member and covering the diaphragm.

In certain embodiments, the sensor is positioned relative to the diaphragm by one or more supports extending from the frame.

In certain embodiments, the system further comprises at least one additional sensor communicatively coupled to the processor. The at least one additional sensor may be selected from a heat sensor, a humidity sensor, a barometric pressure sensor, an ambient noise sensor, an ambient light sensor, an ultrasound sensor, an altitude sensor, a camera, a volatile organic compound sensor, ACG, BCG, ECG, EMG, EOG, SCG, and UTI.

From another aspect, there is provided a method for non-contact monitoring of acoustic signals associated with a body, the method executed by a processor of a system defined in claim 1, the method comprising obtaining vibroacoustic data detected by the sensing device of claim 1 operatively communicable with the processor; extracting, from the detected vibroacoustic signal, a vibroacoustic signal component originating from the subject; and characterizing presence or absence of a bodily condition of the body based at least in part on the extracted vibroacoustic signal component.

In certain embodiments, the diaphragm comprises a compliant material. In certain embodiments, the sensor is positioned relative to an aperture defined by the frame and is connected to the frame. In certain embodiments, the sensor is connected to the frame by at least one edge of the diaphragm and by a magnet housing. The diaphragm may be configured to cover the aperture of the frame. In certain embodiments, the sensing device further comprises a back cover covering the aperture of the frame and spaced from the diaphragm.

In certain embodiments, the sensor is a first sensor, the sensing device further comprises: a second sensor for sensing vibroacoustic signals, the first and second sensors configured to detect acoustic signals having a bandwidth ranging from 0.01 Hz to 160 kHz and each comprising: a voice coil component comprising a coil holder supporting wire windings; a magnet component comprising a magnet supported by a magnet housing, the magnet having a magnet gap configured to receive at least a portion of the voice coil component in a spaced and moveable manner; a connector connecting the voice coil component to the magnet component, the connector being compliant and permitting relative movement of the voice coil component; a diaphragm configured to induce a movement of the voice coil component in the magnet gap responsive to incident acoustic signals, wherein the diaphragm is attached to the voice coil component and the wire windings are spaced from the diaphragm; a frame defining an aperture for holding the first and second sensors, the aperture being at least partially covered by the diaphragm of the first and second sensors, the first and second sensors being connected to the frame such that the diaphragm faces the at least a part of a body of the subject in use.

In certain embodiments, the sensor is a first sensor, the sensing device further comprises: a second sensor for sensing vibroacoustic signals, the first and second sensors for detecting acoustic signals having a bandwidth ranging from 0.01 Hz to 160 kHz and comprising: a voice coil component comprising a coil holder supporting wire windings; a magnet component comprising a magnet supported by a magnet housing, the magnet having a magnet gap configured to receive at least a portion of the voice coil component in a spaced and moveable manner; a connector connecting the voice coil component to the magnet component, the connector being compliant and permitting relative movement of the voice coil component; a diaphragm configured to induce a movement of the voice coil component in the magnet gap responsive to incident acoustic signals, wherein the diaphragm is attached to the voice coil component and the wire windings are spaced from the diaphragm, a frame defining a first aperture for housing the first sensor and a second aperture for housing the second sensor, the first and second apertures being at least partially covered by the diaphragm of the first and second sensors, the first and second sensors being positioned in the frame such that the diaphragm faces the at least a part of a body of the subject in use. The diaphragm of the first and second sensors are connected to the frame. The first aperture and the second aperture may be different sizes.

In certain embodiments, the sensor of the sensing device, instead of or in addition to being a voice coil sensor comprises an Inertial Measurement Unit (IMU) mounted to the diaphragm.

In certain embodiments, the system further comprises a heat sensor for sensing a temperature of the at least a part of the body of the subject in use, and wherein the processor is configured to: receive, from the heat sensor, temperature data corresponding to the subject and collected by the heat sensor; and output, based on the received vibroacoustic signal data, the ultrasound signal data and the heat sensor and using a trained machine learning model, an indication of the presence or absence of the condition in the subject.

In certain embodiments, the system further comprises an environmental sensor configured to detect one or more of an ambient temperature, a barometric pressure, an altitude, ambient noise, and ambient light; and wherein the processor is configured to receive, from the environmental sensor, the one or more of the ambient temperature, the barometric pressure, the altitude, the ambient noise, and the ambient light corresponding to an environment around the sensing device; and calibrate one or both of the received vibroacoustic signal data and the ultrasound signal data based on the one or more of the ambient temperature, the barometric pressure, the altitude, the ambient noise, and the ambient light corresponding to an environment around the sensing device.

In certain embodiments, a ratio of an inductance and moving mass of the vibroacoustic sensor is at least 6.5 mH per gram at 1 kHz. In certain embodiments, a ratio of a mechanical compliance of the connector of the vibroacoustic sensor and moving mass of the vibroacoustic sensor is at least 0.3 mm/N per gram. In certain embodiments, a ratio of a BL product and moving mass of the vibroacoustic sensor is at least 16 N/Amp per gram. In certain embodiments, the housing is substantially upright and is configured to be supported by a wall, a floor or a ceiling. In certain embodiments, the housing has an arch-like configuration including at least one substantially upright portion including the front side and sized so that the subject can stand under the housing. In certain embodiments, the front side of the housing includes a display for displaying information to the subject.

In certain embodiments, the system further comprises an additional sensor, such as a contextual sensor, configured to measure one or more of: optical data, GPS, motion, humidity, pressure, ambient temperature, body temperature, light, sound, radiation, pulse, bioimpedance, skin conductance, galvanic skin response, electrodermal response, and electrodermal activity. The additional sensor data from the additional sensor may be used to which may be environmental/social determinants of health data.

In the context of the present specification, unless expressly provided otherwise, vibroacoustic refers to vibrations and/or acoustical signals propagating through air, biological structures, solids, gases, liquids, or other fluids. This term also encompasses the term mechano-acoustic.

In the context of the present specification, unless expressly provided otherwise, by “body” is meant (i) a living subject, such as a human or animal, or (ii) a non-living object such as a man-made structure (e.g. building, bridge, dam, power generator, turbine, battery, heating/ventilation/air conditioning (HVAC) systems, internal combustion engines, jet engines, aircraft wing, environmental infrasound, ballistics, drones and/or seacrafts, nuclear reactors etc).

In the context of the present specification, unless expressly provided otherwise, by animal is meant an individual animal that is a mammal, bird, or fish. Specifically, mammal refers to a vertebrate animal that is human and non-human, which are members of the taxonomic class Mammalia. Non-exclusive examples of non-human mammals include companion animals and livestock Animals in the context of the present disclosure are understood to include vertebrates. The term vertebrate in this context is understood to comprise, for example fishes, amphibians, reptiles, birds, and mammals including humans. As used herein, the term “animal” may refer to a mammal and a non-mammal, such as a bird or fish. In the case of a mammal, it may be a human or non-human mammal Non-human mammals include, but are not limited to, livestock animals and companion animals.

In the context of the present specification, unless expressly provided otherwise, by “remote” or “contact-free” is meant that certain components of the system do not have direct contact with the body. “Remote” or “contact-free” includes situations in which certain components of the system are spaced from the body, such as by air. There is no limitation on a distance of the spacing. “Remote” or “contact-free” in the context of embodiments of the present system includes signal detection “over clothing” and/or “through clothing”. For example, if the body is a human or animal subject, “remote” or “contact-free” means that certain components of the sensor system do not directly contact the skin/hair, clothing covering the skin/hair or fur.

In the context of the present specification, unless expressly provided otherwise, by “bodily condition” is meant a health or physical condition of a body. For non-living bodies, the bodily condition may include a physical state of the body for example a structural integrity, crack development, battery life, environmental noise pollution, rotating motor engine performance optimization, surveillance etc. For living bodies, the bodily condition may refer to, but is not limited to, one or more of: an identity of the human or animal, a category of the human or animal, a viral infection, a bacterial infection, a heart beat, chest pain and underlying causes, an inhale, an exhale, a cognitive state, a reportable disease, a fracture, a tear, an embolism, a clot, swelling, occlusion, prolapse, hernia, dissection, infarct, stenosis, hematoma, edema, contusion, osteopenia and presence of a foreign body in the subject such as an improvised explosive device (IED), surgically implanted improvised explosive device (SIIED), and/or body cavity bomb (BCB). Examples of viral infections include but are not limited to infections of Covid-19, SARS, influenza. Reportable diseases are diseases considered to be of great public health importance and include: Anthrax, Arboviral diseases (diseases caused by viruses spread by mosquitoes, sandflies, ticks, etc.) such as West Nile virus, eastern and western equine encephalitis, Babesiosis, Botulism, Brucellosis, Campylobacteriosis, Chancroid, Chickenpox, Chlamydia, Cholera, Coccidioidomycosis, Cryptosporidiosis, Cyclosporiasis, Dengue virus infections, Diphtheria, Ebola, Ehrlichiosis, Foodborne disease outbreak, Giardiasis, Gonorrhea, Haemophilus influenza, invasive disease, Hantavirus pulmonary syndrome, Hemolytic uremic syndrome, post-diarrheal, Hepatitis A, Hepatitis B, Hepatitis C, HIV infection, Influenza-related infant deaths, Invasive pneumococcal disease, Lead-elevated blood level, Legionnaire disease (legionellosis), Leprosy, Leptospirosis, Listeriosis, Lyme disease, Malaria, Measles, Meningitis (meningococcal disease), Mumps, Novel influenza A virus infections, Pertussis, Pesticide-related illnesses and injuries, Plague, Poliomyelitis, Poliovirus infection, nonparalytic, Psittacosis, Q-fever, Rabies (human and animal cases), Rubella (including congenital syndrome), Salmonella paratyphi and typhi infections, Salmonellosis, Severe acute respiratory syndrome-associated coronavirus disease, Shiga toxin-producing Escherichia coli (STEC), Shigellosis, Smallpox, Syphilis, including congenital syphilis, Tetanus, Toxic shock syndrome (other than streptococcal), Trichinellosis, Tuberculosis, Tularemia, Typhoid fever, Vancomycin intermediate Staphylococcus aureus (VISA), Vancomycin resistant Staphylococcus aureus (VRSA), Vibriosis, Viral hemorrhagic fever (including Ebola virus, Lassa virus, among others), Waterborne disease outbreak, Yellow fever, Zika virus disease and infection (including congenital). Examples of underlying causes behind chest pain which may be considered as a bodily condition include one or more of muscle strain, injured ribs, peptic ulcers, gastroesophageal reflux disease (GERD), asthma, collapsed lung, costochondritis, esophageal contraction disorders, esophageal hypersensitivity, esophageal rupture, hiatal hernia, hypertrophic cardiomyopathy, tuberculosis, mitral valve prolapse, panic attack, pericarditis, pleurisy, pneumonia, pulmonary embolism, heart attack, myocarditis, angina, aortic dissection, coronary artery dissection, pancreatitis, and pulmonary hypertension.

In the context of the present specification, unless expressly provided otherwise, a computer system may refer to, but is not limited to, an “electronic device”, an “operating system”, a “communications system”, a “system”, a “computer-based system”, a “controller unit”, a “control device” and/or any combination thereof appropriate to the relevant task at hand.

In the context of the present specification, unless expressly provided otherwise, the expression “computer-readable medium” and “memory” are intended to include media of any nature and kind whatsoever, non-limiting examples of which include RAM, ROM, disks (CD-ROMs, DVDs, floppy disks, hard disk drives, etc.), USB keys, flash memory cards, solid state-drives, and tape drives.

In the context of the present specification, a “database” is any structured collection of data, irrespective of its particular structure, the database management software, or the computer hardware on which the data is stored, implemented or otherwise rendered available for use. A database may reside on the same hardware as the process that stores or makes use of the information stored in the database or it may reside on separate hardware, such as a dedicated server or plurality of servers.

In the context of the present specification, unless expressly provided otherwise, the words “first”, “second”, “third”, etc. have been used as adjectives only for the purpose of allowing for distinction between the nouns that they modify from one another, and not for the purpose of describing any particular relationship between those nouns.

Variations of the present technology each have at least one of the above-mentioned object and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects and advantages of embodiments of the present technology will become apparent from the following description, the accompanying drawings and the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A to 1C depict schematic illustrations of a system for characterizing a bodily condition of a subject. FIG. 1D illustrates various types of vibroacoustic data across a range of frequencies, energy distributions, and amplitudes in relation to the human ear's sensitivity across the range of frequencies.

FIG. 2A depicts an exploded view of a sensing device embodied as a panel, according to certain embodiments of the present invention. FIG. 2B depicts front and perspective views of the sensing device of FIG. 2A. FIG. 2C is a cross-section of the sensing device of FIG. 2A.

FIG. 3A depicts an assembled view of an example sensor including a voice coil having one or more spider layers, according to embodiments of the present technology. FIG. 3B is an exploded view of the sensor of FIG. 3A, and having a single layer spider. FIG. 3C is an exploded view of the sensor of FIG. 3A, and having a double layer spider. FIG. 3D is a perspective view of the sensor of FIG. 3A with an outer housing omitted for clarity. FIG. 3E is an exploded view of the vibroacoustic sensor of FIG. 3D.

FIGS. 4A and 4B are cross-sectional views of the example sensors of FIGS. 3A and 3B, respectively.

FIGS. 5A-5AB are example spiders for use with variants of the sensors of any of FIGS. 3A-E, and 4A-B.

FIGS. 6A and 6B depict an example electric potential sensor for use in the system of any of FIGS. 1A-1C, according to certain embodiments of the present technology. FIG. 6B depicts a top plan view of the electric potential sensor and FIG. 6A shows a cross-section through the electric potential sensor. FIG. 6C depicts the example electric potential sensor of FIGS. 6A and 6B in the sensing device of FIG. 2A, according to certain embodiments of the present technology.

FIG. 7 depicts a flowchart summarizing an example method for characterizing a bodily condition of a subject, according to certain embodiments of the present technology.

FIGS. 8A-8C show vibroacoustic test data collected by the sensing system of FIGS. 1A-1C and the sensing device of FIG. 2A, when a subject wearing a sweater is positioned 10 cm from a diaphragm of the sensing device and is facing the diaphragm (FIG. 8A), when the subject wearing a sweater is positioned 10 cm from a diaphragm of the sensing device and is facing away from the diaphragm (FIG. 8B), and when the subject wearing a sweater is positioned 100 cm from a diaphragm of the sensing device and is facing the diaphragm (FIG. 8C).

### DETAILED DESCRIPTION

Non-limiting examples of various aspects and variations of the invention are described herein and illustrated in the accompanying drawings.

### 1. Systems

1.a. Overview

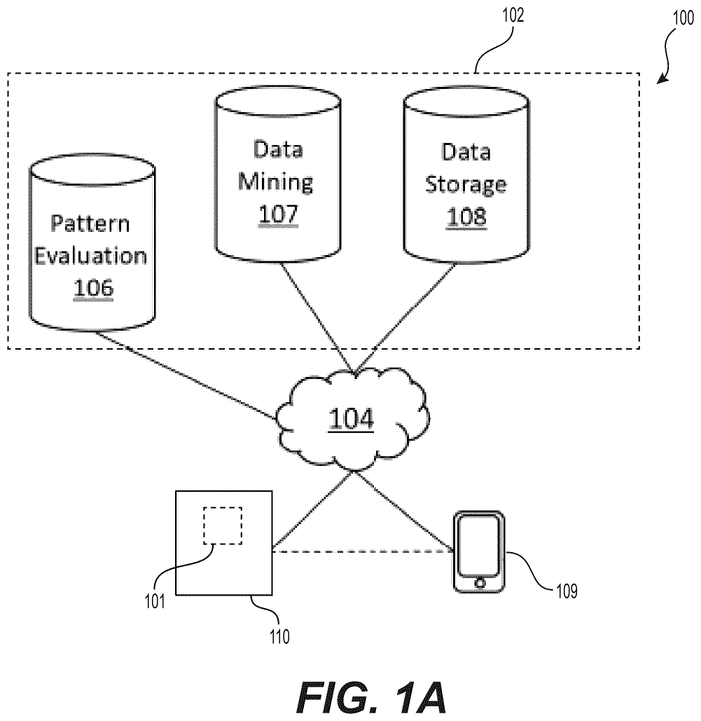

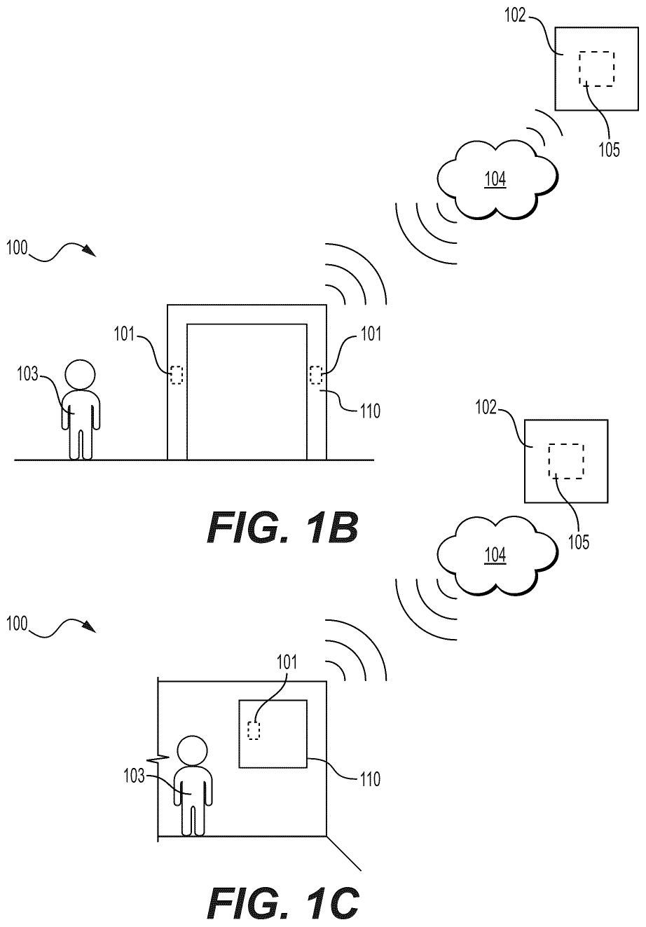

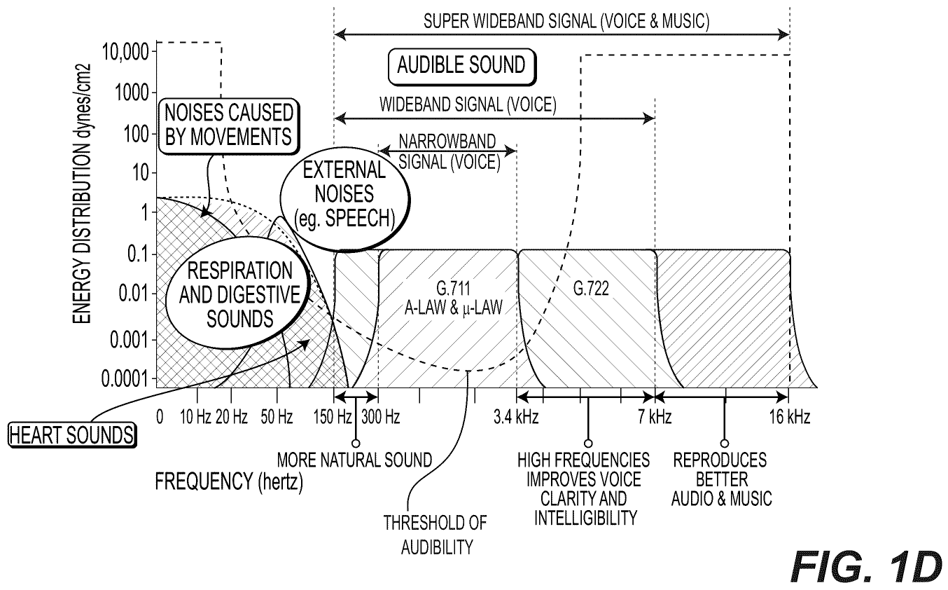

As shown in FIGS. 1A-1C, according to certain embodiments, a system 100 for monitoring a body 103 or characterizing a bodily condition comprises a sensing device 110 having one or more sensors 101 configured to detect one or more parameters associated with the body 103 without contact with the body 103, and a computing system 102, communicatively coupleable to the sensing device 110, and including a processor 105 for receiving sensor data from the sensing device 110 and processing, analyzing, communicating, and/or storing the sensor data/processed sensor data. The computing system 102 may be configured to determine a bodily condition of the body based on the sensor data. As depicted in FIGS. 1A-1C, the system 100 in certain embodiments comprises a single sensing device 110. In other embodiments, the system 100 comprises a plurality of sensing devices 110, each of which may be configured to detect the same or different parameters. The sensing device 110 may include a plurality of sensors 101 (FIG. 1B) or a single sensor 101 (FIG. 1C).

As will be described further below, in certain embodiments, the sensor 101 is configured to detect vibroacoustic signals associated with a body which is a living subject, such as a human or animal subject. However, it will be appreciated that embodiments of the present technology are also applicable to non-living bodies.

In certain embodiments, the sensor 101 is configured to detect vibroacoustic signals within an overall bandwidth ranging from infrasonic, through acoustic, to ultrasonic. In certain embodiments, the bandwidth ranges from about 0.01 Hz to at least about 50 kHz, from about 0.01 Hz to at least about 60 kHz, from about 0.01 Hz to at least about 70 kHz, from about 0.01 Hz to at least about 80 kHz, from about 0.01 Hz to at least about 90 kHz, from about 0.01 Hz to at least about 100 kHz, from about 0.01 Hz to at least about 110 kHz, from about 0.01 Hz to at least about 120 kHz, from about 0.01 Hz to at least about 130 kHz, from about 0.01 Hz to at least about 140 kHz, from about 0.01 Hz to at least about 150 kHz, from about 0.01 Hz to at least about 160 kHz, from about 0.01 Hz to more than about 150 kHz.

As mentioned above, Developers have noted that frequencies within both the non-audible (e.g. infrasonic and ultrasonic) and audible ranges are useful in determining the bodily condition. As shown in FIG. 1D, the threshold of human audibility decreases sharply as vibrational frequency falls below about 500 Hz. However, in a healthy subject at rest, most cardiac, respiratory, digestive, and movement-related information is inaudible to humans, as this information occurs at frequencies below those associated with speech. Thus, the majority of bodily vibrations are neither detected nor included in conventional diagnostic medical practices due to the low frequency band of these vibrations, and the limited bandwidth limits of conventional instruments (e.g., conventional stethoscopes). Variations of the system 100 described herein are capable of detecting, amplifying and analyzing a broad spectrum of infrasound, ultrasound, and far-ultrasound vibroacoustic frequencies, and are thus advantageous for a more comprehensive, holistic picture of subject health and condition. In addition, embodiments of the system 100 are able to detect signals across this broad bandwidth with sufficient sensitivity to be able to process the signals and to detect a bodily condition.

The sensing device 110 may have any suitable form factor for detecting parameters of the body in a non-contact manner. The sensing device 110 may be configured and positionable in any suitable manner relative to the body to capture the suitable parameter(s) in a contact-manner. For example, the sensing device 110 may be configured to be supported by a support surface, such as free-standing on a floor (FIG. 1B), or mounted to a wall or ceiling (FIG. 1C). In other embodiments (not shown), the sensing device 110 may be integrated into furnishings and other structures such as cabinets, fridges, freezers, light fixtures, mirrors, panels, kiosks, doorways, signs, fitness equipment, security gates, security arches, home security systems, ticket machines, etc.

The computing system 102 may be separate from the sensing device 110, or be incorporated within the sensing device 110. The computing system 102 may also be partially incorporated in the sensing device 110 and partially remote thereto. The computing system 102 may be embodied in any form such as but not limited to a server, a mobile computing device, a personal computer, or a local data gateway. In some variations, the computing device 102 may be implemented as a network-on-chip (NoC) technology. The computing system 102 may be configured to receive data from the sensor 101 or the sensing device 110 and use the sensor data in the processing, analyzing, communicating, and/or storing functions. The computing system 102 may additionally collect data from other sensors, such as scales, contextual sensors, cameras, thermometers, that can provide supplementary environmental and social determinants of health contextual information.

As shown in FIGS. 1A-1C, the sensing device 110 may be configured to communicate wirelessly over a network 104 with the computing system 102. Additionally or alternatively, the sensing device 110 may be configured to communicate directly with the computing system 102 without the network 104 (e.g., in pairwise fashion). In other variations, the sensing device 110 may be configured to communicate directly with the network 104.

Referring to FIG. 1A, in some embodiments, the computing system 102 comprises one or more modules such as, for example, (i) a pattern evaluation module 106 which may incorporate artificial intelligence (e.g., through application of one or more trained machine learning models) to characterize one or more bodily conditions of the subject based on the sensor data from the sensing device; (ii) a data storage module 108 for storing the sensor data or processed sensor data, the other data from the sensors (if applicable) and/or electronic medical records associated with the subject; and (iii) a data mining module 107 for use in training and increasing the accuracy of predictive and/or prescriptive models across patient populations. A communication module (not shown) may be provided for communicating data between the various modules of the system. The communication module, or another module, may also be provided for communicating information to an operator of the system 100 (e.g. an entity who desires to be informed of the determined bodily condition), or to the subject being monitored. For example, the computing system 102 may be configured to cause an output such as a haptic signal, a display, a light signal, or an audio signal on a device 109 associated with the operator of the system 100 or associated with the subject. The computing system 102 may be configured to execute one or more methods using the signal data, as will be described in further detail below. The communication module may also be responsible for receiving sensor data from the sensor 101 or the sensing device 110.

In certain embodiments, the sensing device 110 may be modular and include interchangeable subsystems adapted for modular experimentation, optimization, manufacture, rapid field configurability, etc. as part of a modular sensing platform. For example, the sensing device 110 may include the one or more sensors 101 which could be interchangeable, an electronics module, and/or other components that may be interchangeable for different applications and contexts. Such a modular sensing platform may provide an architecture well-suited for a modular suite of, for example, remote screening devices and/or point-of-care solutions for healthcare, etc.

1.b. Sensing Device

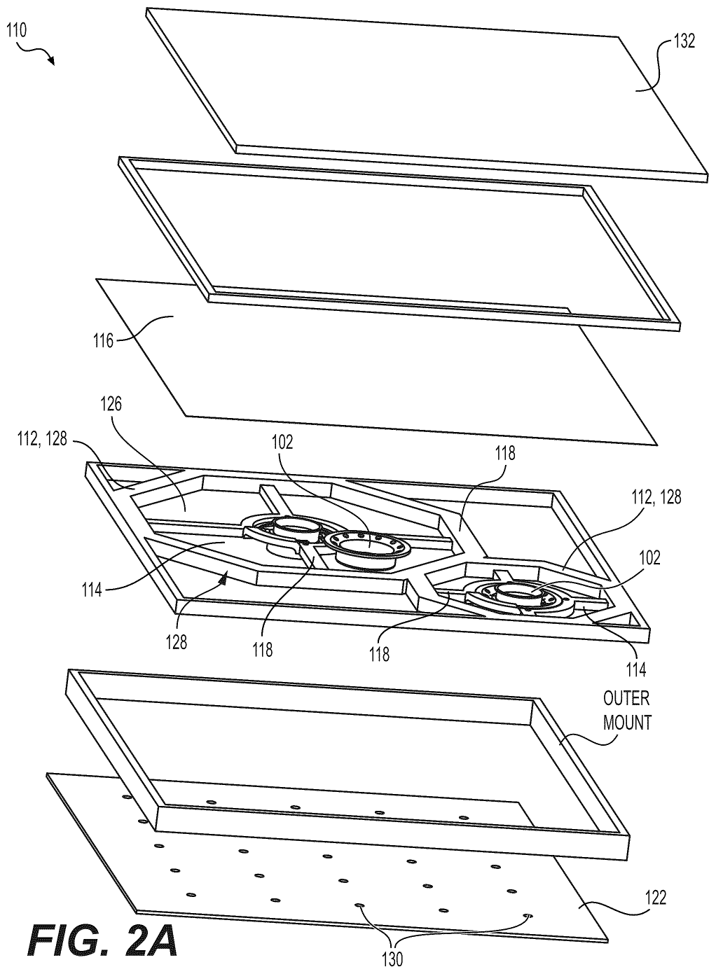

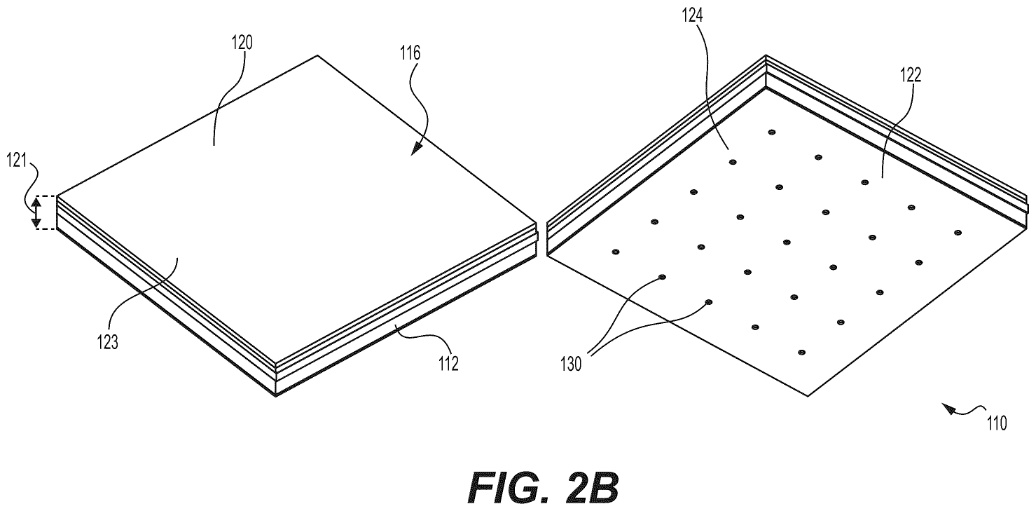

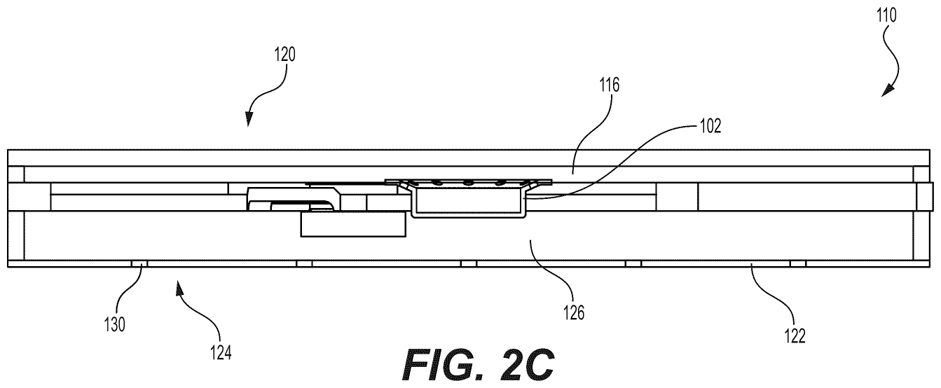

Referring to FIGS. 2A-2C, in certain embodiments, the sensing device 110 has a panel-like form. By panel-like is meant that the sensing device 110 has an outward facing surface (first side 120) which is generally flat and continuous.

The sensing device 110 may be mountable to a support surface such as a wall, floor, a ceiling, a doorway, etc., or be free standing on the support surface. The sensing device 110 may be camouflaged so as not to be apparent to the subject. In this respect, the sensing device 110 may be incorporated within a furnishing such as a cabinet, door, doorway, mirror, fridge, security gate, etc. The sensing device 110 may be installed in rooms, corridors, vehicles, entryways, checkpoints, doorways, vehicles, and other areas to detect sensor signals from subjects for diagnosing certain bodily conditions of the subjects. The sensing device 110 may be part of a security system such as a gateway, door etc., and be used to verify an identity of the body.

In use, in certain embodiments, the sensing device 110 is configured to be positioned such that the first side 120 is configured to face, and be spaced from, at least a portion of the body to detect vibroacoustic signals therefrom. For example, in certain examples, the sensing device 110 may be configured to be positioned substantially vertically so that it faces a torso, head or hand of a human subject who is walking, standing or sitting. In other examples, the sensing device 110 may be configured to be positioned substantially horizontally so that the human subject can wave a hand above it. This particular configuration may be used in embodiments in which the bodily condition comprises an identification of the subject and implemented in security uses.

The sensing device 110 may also have a thin-form in that a depth 121 of the sensing device 110 is less than a surface area 123 of the first side 120. In certain embodiments, the depth 121 is less than 30 cm, 25 cm, 20 cm, 15 cm, 10 cm, 7.5 cm, 5 cm, 4 cm, 3 cm, 2 cm, or 1 cm. In certain embodiments, the depth of the sensing device 110 may be less than 1 mm, 2 mm, 3 mm, 4 mm, 5 mm, 6 mm, 7 mm, 8 mm, 9 mm or 10 mm. In certain embodiments, the depth 121 of the sensing device 110 is delimited by a thickness of the sensor 101.

In certain embodiments, the surface area 123 of the first side 120 is related to a required sensitivity as the size of the first side 123 will determine a size of a diaphragm 116 either forming the first side 123 or positioned beneath. In certain embodiments, a diameter or a largest width of the first side 120 is less than about 100 cm, 90 cm, 80 cm, 70 cm, 60 cm, 50 cm, 40 cm, 30 cm, 20 cm, 10 cm, 9 cm, 8 cm, 7 cm, 6 cm, or 5 cm. A minimum diameter is estimated to be 0.5 cm, resulting in about 0.2 square cm area.

The sensing device 110 is configured to operate at any distance from the body, such as but not limited to: 1 cm, 5 cm, 10 cm, 25 cm, 50 cm, 100 cm, 150 cm, 200 cm, 250 cm, or 300 cm.

Referring more specifically to FIGS. 2A and 2C, the sensing device 110 comprises a support member, such as a frame 112, defining an aperture 114, and the diaphragm 116 extending at least partially across the aperture 114 and supported by the frame 112. The diaphragm 116 is configured to vibrate freely in at least some portion(s). The sensor 101 comprises a vibroacoustic sensor, which is coupled to the frame 112 such as by at least one support 118. In certain embodiments, there are provided a plurality of support members 117 which serve to connect the sensor 101 to the frame and position the sensor 101 relative to the aperture 114. As best seen in FIG. 2A, the support includes a circular portion to which the sensor 102 is attached, and strut portions extending from the circular portion to the frame 112.

The diaphragm 116 is configured to vibrate at frequencies relating to a biovibroacoustic range of the subject. The sensor 101 is configured to convert vibrations of the diaphragm 116, such as to an analog or digital signal. The sensor 101 can be any type of sensor which can convert diaphragm 116 movement to an electrical signal, such as but not limited to a voice coil-type transducer, an electric potential sensor, a capacitive sensor, an accelerometer, and combinations of the same.

### Support Member

The support member 112 may be of any suitable size or shape, the dimensions and configuration of which are selected based on the desired use and the desired frequency range of detection. The support member 112 may be constructed from any suitable material such as plastic, wood, metal, composite, glass, ceramic, or any other suitable material that can withstand the tension of the attached diaphragm and/or support the attached diaphragm 116. Although illustrated as an octagonal frame, the support member 112 can be any shape such as circular, oval, rectangular, trapezoidal, regular polygonal, or non-regular polygonal. The support member 112 can be of any size. In certain embodiments, the support member 112 may be a component of the support surface or a furnishing.

A thickness of the support member 112 is not limited. For example, a width of the support member 112 is less than a width of the aperture. In other embodiments, the width of the support member 112 may be more wide than a width of the aperture.

As mentioned previously, the support member 112 defines the aperture 114 associated with the sensor 101. The support member 112 may be configured to define the aperture 114 so that it extends through the support member 112 (i.e. the aperture is open on both the first side 120 and the second side 124). In these embodiments, the support member 112 is referred to herein as a “frame 112”. In other embodiments (not shown), the support member 112 may define the aperture in only one of its sides, i.e. the first side 120 or the second side 124.

As illustrated, in certain embodiments, the sensing device 110 is configured to provide a plurality of frames, herein referred to as sub-frames 128 as they share common frame portions. Each sub-frame defines a respective aperture 114, with each aperture 114 associated with a separate sensor 101. This configuration can also be applied to the embodiments in which the aperture 114 does not extend through the support member 112. An outer mount may be provided for structural integrity. This can be seen in the figures as the rectangular outer frame but it will be appreciated that it is optional. When provided, the diaphragm 116 may be attached to the outer mount as well as the support member 112, particularly when the diaphragm 116 does not seal the aperture 114.

In the embodiments illustrated, there are provided two sensors 101, housed within different sub-frames 128. The sub-frames 128 are adjacent one another and have a sub-frame portion in common, in certain embodiments. In this respect, the sensing device 110 may be considered to comprise multiple transducers operating on the same or different modes of operation. The multiple transducers may be arranged as an array.

In certain embodiments, the sub-frames 128 share the same diaphragm 116. In other words, there is provided a single diaphragm 116 attached to the frame 112 and covering the sub-frames 128. The diaphragm 116 can be attached to the sub-frames 128 to provide a hinging effect.

In certain embodiments, the sub-frames 128 each have a respective diaphragm 116. In other words, there are a plurality of diaphragms 116 provided per frame 112, one diaphragm 116 per sub-frame 128.

In either scenario, various parameters may be tailored to tailor the frequency range detection, such as one or more of the sensor 101 pick-up range, the diaphragm 116 surface area, diaphragm stiffness, and diaphragm weight, any weight or damping added to the diaphragm (such as attaching the sensor 101 thereto).

In certain embodiments, the sub-frames 128 can be of different sizes, for example a first sub-frame 128 may be larger than a second sub-frame. In such an embodiment, the first sub-frame 128 and its associated larger area of diaphragm 116 may be able to detect vibroacoustic signals which are <20 Hz, whereas the second sub-frame and its respective diaphragm 116 may be configured to detect >100 Hz.

In certain embodiments, the sub-frames 128 may be separate and spaced from one another within the frame 112. In other words, the sub-frames may not share a common sub-frame portion. The sensing device 110 could still be considered to comprise multiple transducers operating on the same or different modes of operation. The multiple transducers may be arranged as an array.

In certain embodiments, the sensing device 110 may comprise a plurality of support members 112 (which may or may not include sub-frames), each support member 112 defining a respective aperture, and having the respective sensor 101 attached thereto. Again, the multiple transducers (sensors 101) may be considered as an array. Each support member 112 of the plurality of frames 112 may be configured to detect vibroacoustic signals of a differing range to thus provide an overall bandwidth of detected signals across the plurality of frames 112 which is broader than that of an individual support member 112 within the plurality of support members 112. The bandwidth of vibroacoustic signal detectable by each support member 112 may be tailored, in certain embodiments, by

Sensing devices 110 without sub-frames 128 as well as sensing devices 110 with sub-frames 128 are within the scope of the present technology. Sub-frames 128 may be provided in sensing devices 112 in which the support member 112 defines the aperture 114 that does not extend therethrough. It will be appreciated that the configuration of the sub-frames may differ from the configuration as illustrated, in a manner known by persons skilled in the art.

1.c. Diaphragm

In certain embodiments, the diaphragm 116 is positioned at the first side 120 of the sensing device 110. In embodiments in which the support member 112 is a frame, a back cover 122 may be provided on the second side 124 of the sensing device 110, thereby defining a cavity 126 between the diaphragm 116 and the back cover 122. The diaphragm 116 is configured to vibrate at frequencies relating to a desired detection frequency range, such as the vibroacoustic range of the subject. One or more of the parameters of the material, weight, size and tension of the diaphragm 116, as well as the shape or size of the cavity 126 behind the diaphragm 116, may be tailored to achieve the desired frequency range.

In some embodiments, the diaphragm 116 may generally have a nominal or resting configuration in which the diaphragm 116 is arranged in a plane, and the diaphragm 116 may deflect or flex in response to out-of-plane forces. In these variations, the diaphragm 116 may be configured to have low stiffness (or resistance) against out-of-plane movement with good compliance to acoustic movement, yet high stiffness or resistance against in-plane movement and low crosstalk between axes within the plane. Accordingly, the diaphragm 116 may have high sensitivity to acoustic waves directed toward the diaphragm 116 (that is, acoustic waves having a vector component that is orthogonal to the deflecting structure) but be robust against noise contributed by other forces.

Furthermore, in some variations, the diaphragm 116 may have relatively low mass on a movable portion of the deflecting structure to reduce inertia (and further improve sensitivity to out-of-plane forces). In some variations, the deflecting structure may be designed with low or no hysteresis, such that out-of-plane movement is highly linear.

Larger diaphragms 116 with low stiffnesses tend to pick up low frequencies well, whereas stiffer diaphragms 116 pick up higher frequencies but attenuate lower ones. The weight of the diaphragm 116 itself or anything connected to the diaphragm 116 in general causes inertia during vibrations, which oppose and attenuate incoming vibroacoustic signals (and might cause increased reflection of the acoustic wave).

In embodiments in which the sensor 101 is a voice coil transducer, as it is connected to the diaphragm 116 and has deflecting components, this can provide an additional spring in the system; which adds to the diaphragm 116 stiffness and decreases the compliance of the sensor pickup. The attached voice coil portion may also add inertia to the diaphragm 116.

For example, more compliant diaphragms 116 give good signal-to-noise ratio favoring low frequencies (e.g. 0-100 Hz only). Similarly, larger diaphragms 116 favor lower frequencies as well Smaller diaphragms 116 can detect high bandwidth or higher frequencies. Thicker diaphragm 116 can detect high bandwidth, higher frequencies due to generally higher membrane bending stiffness. Thinner diaphragms 116 can detect lower frequencies as they tend to be more compliant if all other parameters equal. Higher tension diaphragms 116 can detect high bandwidth, less deflection which may lead to lower sensor amplitudes and hence signal-to-noise ratio. Lower tension diaphragms 116 can detect lower bandwidth as more compliant, high deflection caused by same incoming acoustic wave (good signal-to-noise ratio).

Generally, a tradeoff is required between different values of the bending stiffness and hence ability to pick up low amplitude waves. Low bending stiffness results in a compliant diaphragm 116 is able to pick up waves of very low amplitudes (e.g. when <20 Hz). However, the resonance frequency and subsequent roll-off of a very compliant diaphragm 116 is very low and hence obstructing the ability to pick up higher frequencies, particularly above some threshold frequencies, e.g. >100 Hz. High bending stiffness in contrast results in higher resonance modes of the diaphragm 116 giving the ability to pick up higher frequencies at the expense of small amplitude lower frequencies.

In certain embodiments, as an alternative to finding a trade-off for an overall frequency range, the sensing device 110 can be divided into the smaller sub-frames 128 discussed above, each sub-frame 128 having its respective sensor 101. The sub-frames 128 have different diaphragms 116 attached thereto, the different diaphragms 116 configured for a particular frequency range by tailoring one or both of the sensor 101 or the diaphragm 116 stiffness, weight etc. In this manner, by using sub-frames 128, a broader overall frequency range may be detected.

In certain embodiments, the diaphragm 116 is a compliant material such as a thermoplastic or thermoset elastomer. In other embodiments, the diaphragm 116 may comprise metal, inorganic material such as silica, alumina or mica, textile, fiberglass, Kevlar™, cellulose, carbon fiber or combinations and composites thereof. In certain embodiments, the diaphragm 116 is provided with a protective layer which may comprise an acoustically transparent layer, such as foam, positioned on an outer facing side of the diaphragm at any distance, such as from about 1 mm to about 100 mm.

The diaphragm 116 may be attached to the support member 112 in any manner, such as by adhesive. A profile of the diaphragm 116 when attached to the support member 112 may be planar, convex or concave. If the diaphragm 116 is under tension, it may be attached to the support member 112 in a manner to apply a homogenous tension or different tensions along different orthogonal axes. The diaphragm 116 may be a stretched sheet. The diaphragm 116 may, in certain variations, be self-supporting or under compression instead of under tension. A damping material may be provided to dampen movement of the membrane.

With respect to the cavity 126, certain variants of the sensing device 110 provide differing extents of sealing of the cavity 126 by the back cover 122. For example, in certain embodiments, the back cover 122 may be omitted. In this case, pressure on either side of the diaphragm 116 can equalize quickly. However, a diaphragm 116 can generally only bend/vibrate if there is a difference in pressure between the two sides. Since particularly at low frequencies the air has plenty of time to continuously equalize the pressure on the sides of the diaphragm 116 upon the incoming pressure wave it is impossible to measure such low signals. It is then also obvious that static pressure cannot be measured with an open back setup.

In certain other embodiments, in which the back cover 122 is included on the sensing device 110, the back cover 122 may function to seal the cavity 126 to different extents. At one extreme, the back cover 122 may comprise a solid piece which seals the cavity 126. This can be considered like a pressure sensor which measures static pressure against the inside reference pressure. It measures down to DC (static pressure), but the static pressure opposes diaphragm 116 movement to AC signals particularly the higher the input vibration amplitude. In addition, a completely sealed cavity 126 causes the diaphragm 116 to bend outwards or inwards when outside pressure is not equal to inside pressure, e.g. changing altitude. Result may be low Signal-to-Noise Ratio (SNR) at dynamic (AC) measurements at higher frequencies and larger amplitudes, depending on the volume of the cavity.

In certain other embodiment, the back cover 122 includes openings 130 for permitting air flow therethrough to the cavity 126. The size, count and location of these openings 130 can be optimized according to the desired frequency detection range and acceptable signal-to-noise ratios, and can be also seen as a cavity impedance optimization with the cavity volume itself. For low frequency detection (less than 20 Hz), the low frequency pressure waves give plenty of time for creating an equilibrium on either side of the diaphragm 116. So the configuration of the openings 130 need to take into account a tradeoff between letting air in/out (depending on positive or negative pressure waves) from inside the cavity 126 to reduce pressure, and delaying the equilibrium process long enough to catch very low frequency pressure waves. Hence, the pressure on either side of the diaphragm 116 will equalize at some time constant and vibrations at frequencies corresponding to a time period below that equilibrium time constant can be measured.

It will be appreciated that this also applies to embodiments in which the support member 112 defines the aperture 114 in only one side, in which case the other side functions as the back cover. One or more openings may be provided to help equal pressure in the cavity 126.

In certain embodiments, the sensing device 110 is about 7 inches wide, about 9.75 inches high, and about 0.5 inches deep. However, it will be appreciated that the sensing device 110 may have any other size appropriate to its use. Experimental data obtained with this sensing device example is presented in Example 2.

The openings 130 can have any shape (round, square, rectangular), size and count. The openings 130 can be of structure instead of simple opening, such as tubes of various diameter and lengths like commonly present in acoustic subwoofers. Structures as opening can be anything that allows flow of air between the cavity and outside environment, son not only limited to tubes. In an example embodiment the back cover 122 could have a single small tube to equalize for inside DC pressure in a low frequency optimized panel with a large cavity.

In certain other embodiments, the cavity 126 inside the sensing device 110 can be divided into two lateral sections. The divider between the two cavities is perforated based on design needs to allow for air exchange between the two cavities. In one embodiment, the cavity close to the diaphragm 116 is a smaller one and the cavity towards the back is the bigger one, serving as an air ‘reservoir’. The overall unit is sealed off from the environment entirely, or sealed with a small hole or tube to allow pressure equalization with the environment in case of slow and nearly DC type of pressure changes due to e.g. altitude change.

The dual cavity setup may be useful particularly when the sensor 101 is an electric potential or capacitive sensor. For example, in the capacitive sensing approach, there is provided a conductive plate behind the diaphragm 116 to form the capacitor which may be of similar size as the diaphragm 116 to maximize sensitivity. As the conductive plate should be close to the diaphragm 116 to maximize capacitance between the diaphragm 116 and the conductive plate, the cavity formed is small, causing air pressure to rise under a vibrating membrane when a plate without any perforation is used. Hence, perforation in the conductive plate connects the small cavity to the bigger back cavity for reduced pressure.

Generally, when the sensing device 110 is assembled, the cavity 126 should be generally closed, or fluidly sealed, by one or more of the diaphragm 116, the back cover 122, and the support member 112. The cavity 126 may be closed or fluidly sealed by the diaphragm 116 closing or sealing around the support member 112 (e.g. the sub-frame 128 when present), or around the outer mount, when present. In certain embodiments, the cavity is considered closed but not fluidly sealed when one or more of the openings 130 are provided. Generally, sealing about the outer mount may reduce a force required to displace the membrane and at the same time an incoming acoustic wave is able to exert a larger force on the diaphragm 116 due to the larger diaphragm area than can be made to move.

1.d. Positioning of the Vibroacoustic Sensor Assembly Relative to the Membrane

The sensor 101 can be positioned at any appropriate position with respect to edges of the diaphragm 116. The positioning of the sensor 101 may be achieved by means of the one or more supports 118. The one or more supports 118 may extend from the support member 112 or the sub-frame 128 inwardly into the respective aperture 114 to position the sensor 101 at a given position within the aperture 114.

In certain embodiments, as illustrated, the sensor 101 is positioned centrally with respect to the edges of the diaphragm 116. However, the sensor 101 does not necessarily need to be centered with respect to the diaphragm 116. Particularly in embodiments in which the diaphragm 116 could be excited at higher eigenmodes, there is a benefit of placing the sensor 101 off-center in any appropriate position. For example, if the sensor 101 is placed in the center and a higher eigenmode has a node at the center, there will be no displacement at the center and no signal measured, where in reality the diaphragm 116 is indeed vibrating.

For example, consider the diaphragm 116 having a plurality of eigenmodes based on its geometry which will create nodes (points at which there is no displacement) on the diaphragm 116. For example, if the diaphragm 116 has four eigenmodes with a 2×2 configuration, there will be a node at the center of the diaphragm 116. This is also the case when the diaphragm 116 has two eigenmodes which also create a node (no displacement) at a central portion of the diaphragm 116. In these cases, and other eigenmode situations not described, a centrally positioned sensor 101 is not optimally positioned for detecting vibrations in the diaphragm 116. Accordingly, a positioning of the sensor 101 relative to the diaphragm 116 can be selected by considering the eigenmodes of the diaphragm 116.

In embodiments of the sensing device 110 in which the sensor 101 comprises an electric potential sensor and/or a capacitive sensor, the electrodes of such sensors can be sized to cover the size of the diaphragm 116, which can minimize the localized effects of eigenmodes such as no bending/displacement of the diaphragm 116 at the node. In certain other embodiments, the sensor 101 may be configured to not sense beyond the first membrane resonance caused by the first eigenmode, which may also minimize or make redundant an effect of eigenmodes.

1.e. Front Cover

In certain embodiments, the sensing device 110 may be provided with a front cover 132 provided at the first side 120. The front cover 132 may be more rigid than the diaphragm 116. The front cover 132 may provide environmental and mechanical protection of the diaphragm 116 as it is placed more outwardly than the diaphragm. The front cover 132 may have any type of surface finish or configuration. For example, in certain embodiments, the front cover 132 is highly reflective like a mirror. In certain variants, the front cover 132 may include an output display. The output display may include any manner of markings and indicators such as one or more of: the likelihood of the subject having a given bodily condition (e.g. displayed as a red or green light or other indicator), at least a portion of the data obtained by the sensor 101 (e.g. physiological data of the subject, environmental data). In certain embodiments, the front cover 132 may be at least partially a mirror and at least partially an output display such as the output display. The front cover 132 may be configured to extend substantially vertically when supported on a support surface such as the wall or the floor. The front cover 132 may be perforated to permit sound pressure come through without much attenuation, with the perforation down to micrometer size.

In summary, the vibroacoustic detection range of the sensing device 1100 can be tailored based on various parameters relating to: the diaphragm 116 (e.g. stiffness, material, surface area, etc.), the sensor 101 (e.g. voice coil, capacitive, electric potential, optical, acoustic (echo doppler), radar, etc.), pressure equalization based, for example, on size of the cavity 126 and the openings 130 of the back cover 122.

1.f. Sensors—General

The one or more sensors 101 used in the sensing device 110 is not particularly limited. In certain embodiments, the sensor 101 is a vibroacoustic sensor for detecting vibroacoustic signals associated with the object. In some embodiments, transmission of vibroacoustic waves may occur through an intermediate medium such as air.

In some embodiments, the vibroacoustic sensor may have a bandwidth suitable for detecting vibroacoustic signals in the infrasound range, such as a bandwidth ranging from about 0.01 Hz to at least about 20 Hz. Furthermore, in some embodiments, the vibroacoustic sensor may have wider bandwidths covering a wider spectrum of infrasound-to-ultrasound, such as a bandwidth ranging from about 0.01 Hz to at least 160 kHz. In some embodiments, the biological vibroacoustic signal component extracted from the detected vibroacoustic signal may have a bandwidth ranging from about 0.01 Hz to 0.1 Hz.

For example, in some embodiments the vibroacoustic sensor may have an overall bandwidth ranging from about 0.01 Hz to at least about 50 kHz, from about 0.01 Hz to at least about 60 kHz, from about 0.01 Hz to at least about 70 kHz, from about 0.01 Hz to at least about 80 kHz, from about 0.01 Hz to at least about 90 kHz, from about 0.01 Hz to at least about 100 kHz, from about 0.01 Hz to at least about 110 kHz, from about 0.01 Hz to at least about 120 kHz, from about 0.01 Hz to at least about 130 kHz, from about 0.01 Hz to at least about 140 kHz, from about 0.01 Hz to at least about 150 kHz, from about 0.01 Hz to at least about 160 kHz, from about 0.01 Hz to more than about 150 kHz.

The sensor 101 may, in some embodiments, comprise a single sensor 101 that provides one or more of the abovementioned bandwidths of detected vibroacoustic signals.

In some other embodiments, the sensor 101 may include a suite or array of multiple sensors 101, each having a respective bandwidth range forming a segment of the overall vibroacoustic sensor bandwidth. At least some of these multiple sensors 101 may have respective bandwidths that at least partially overlap in certain embodiments. In other embodiments, the multiple sensors 101 do not have overlapping bandwidth ranges. Accordingly, various sensor bandwidths may be achieved based on a selection of particular sensors that collectively contribute to a particular vibroacoustic sensor bandwidth. In other words, bandwidth extension and linearization approach (bandwidth predistortion) may utilize modular sensor fusion and response feedback information, such as to compensate for bandwidth limitations of any particular single sensor with overlapped combinations of sensors to cover a wider bandwidth with optimal performance.

For example, the sensor 101 may be selected from passive and active sensors for obtaining vibroacoustic data such as one or more of a microphone (e.g. dynamic microphone, a large diaphragm condenser microphone, a small diaphragm condenser microphone, and/or a ribbon microphone), a voice coil, an electric potential sensor, an accelerometer, pressure sensors, piezoelectric transducer elements, doppler sensors, etc. Additionally, or alternatively, the vibroacoustic sensor may include a linear position transducer. Such sensors may be configured to detect and measure vibroacoustic signals by interfacing with the diaphragm 116 that moves in response to a vibroacoustic signal.

Additionally, or alternatively, the sensor 101 may include a MEMS cross-axis inertial sensor fusion capable of detecting vibroacoustic signals ranging from about 1 Hz (or less) to a few kHz (e.g., between about 1 Hz and about 2 kHz). Even further, the sensor 101 may additionally or alternatively include a MEMS cross-axis inertial sensor capable of detecting vibroacoustic signals ranging from about 0.01 Hz to several hundred Hz (e.g., between about 0.05 Hz and about 10 kHz). In some variations, the sensor 101 may combine multiple microelectromechanical systems technologies cross-axis inertial sensors capable of detecting vibroacoustic signals ranging from about 20 Hz to about 20 kHz, when intentionally limited to human auditory range.

In certain embodiments, the sensor 101 is one or more selected from a voice coil type transducer, an electric potential sensor, a capacitive pick up sensor, a magnetic field disturbance sensor, a photodetector, a strain sensor, an acoustic echo doppler.

1.g. Sensors—Voice Coil Type Vibroacoustic Transducer

In certain embodiments, the sensor 101 may be based on a vibroacoustic transducer of a voice coil type. Examples of voice coil transducers have been previously described in PCT/IB2021/053919 filed on May 8, 2021 and PCT/US2021/046566 filed on Aug. 18, 2021, the contents of which are herein incorporated in their entirety.

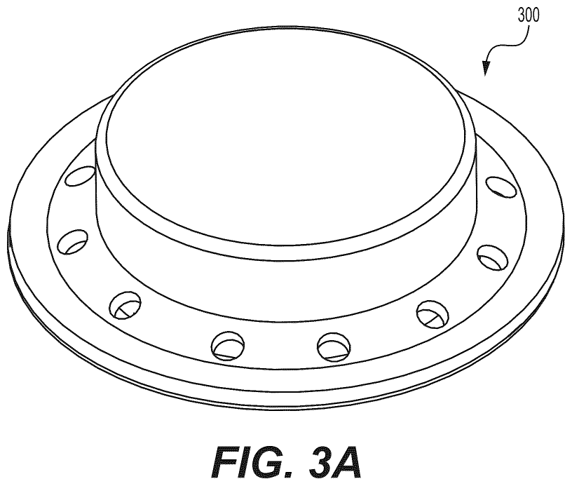

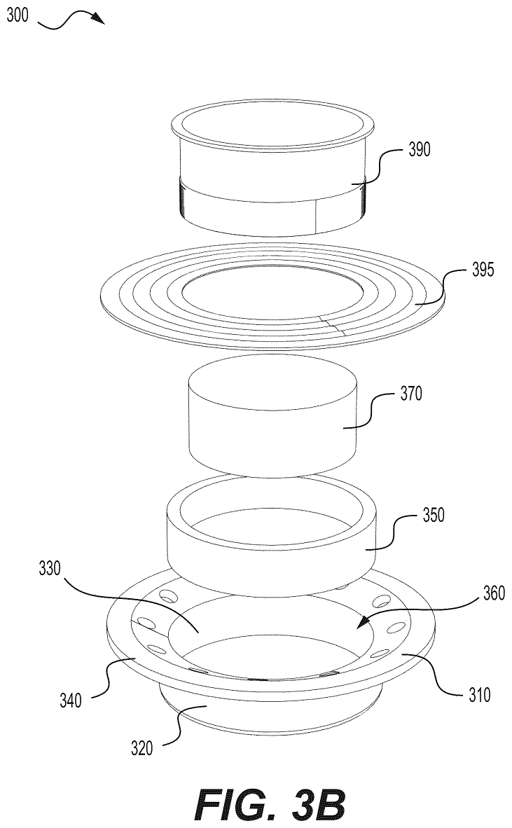

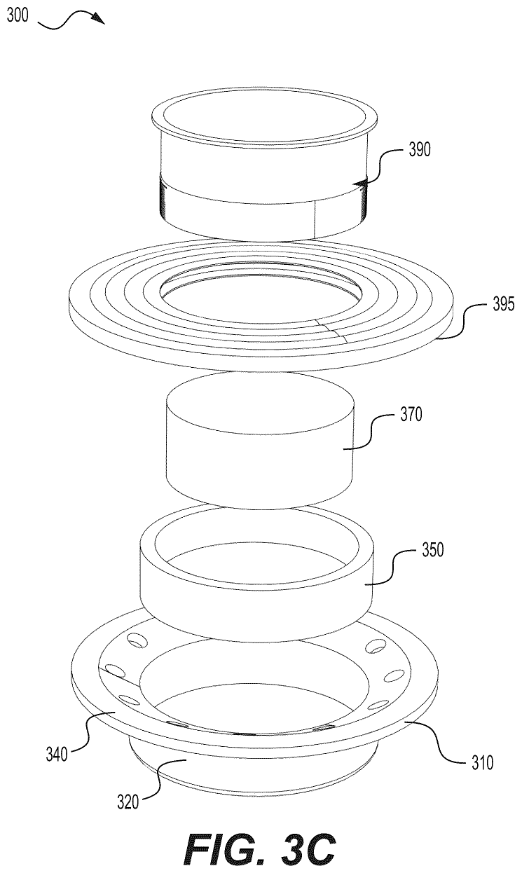

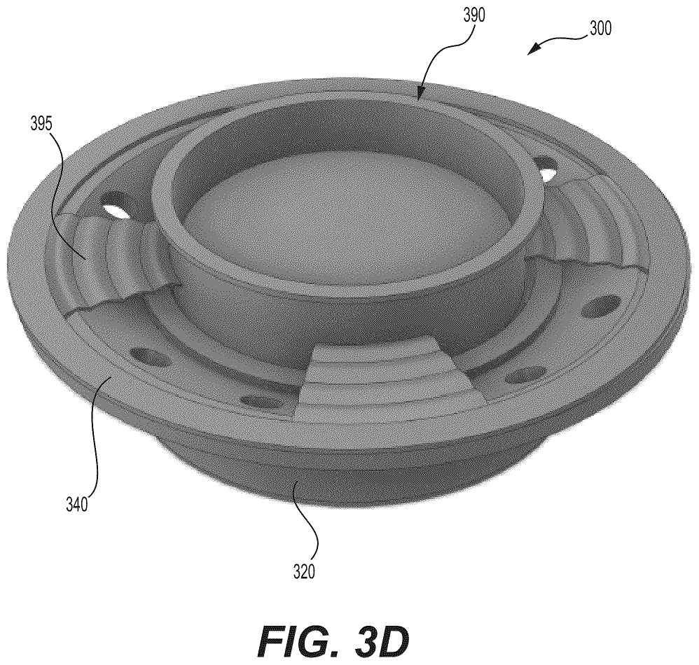

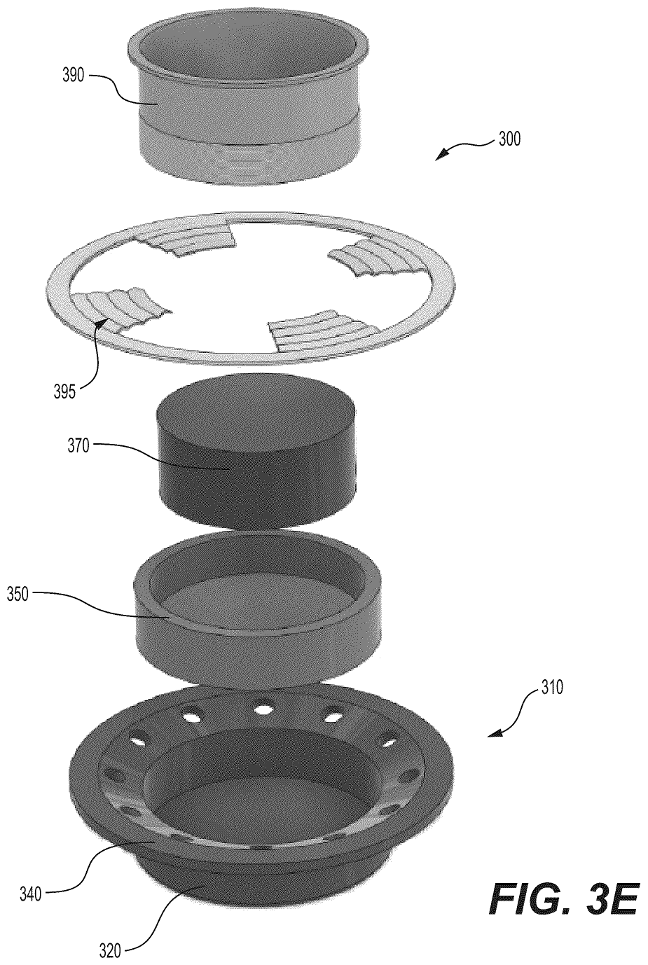

Referring to FIGS. 3A-3E, 4A-4B, and 5A-AB, there is shown the vibroacoustic transducer 300, which is the sensor 101 in certain embodiments, which comprises a frame 310 (also referred to as a magnet housing or a surround pot) having a cylindrical body portion 320 with a bore 330, and a flange 340 extending radially outwardly from the cylindrical body portion 320. The frame 310 may be made of steel. An iron core 350 such as soft iron or other magnetic material is attached to the cylindrical body portion 320 and lines the bore 330 of the cylindrical body portion 320. As can be seen, the iron core 350 extends around the bore 330 of the cylindrical body portion 320 as well as across an end 360 of the cylindrical body portion 320. The iron core 350 has an open end. A magnet 370 is positioned in the bore 330 and is surrounded by, and spaced from, the iron core 350 to define a magnet gap 380. A voice coil 390, comprising one or more layers of wire windings 392 supported by a coil holder 393, is suspended and centered in relation to the magnet gap 380 by one or more spiders 395. The wire windings 392 may be made of a conductive material such as copper or aluminum. A periphery of the spider is attached to the frame 310, and a center portion is attached to the voice coil 390. The voice coil 390 at least partially extends into the magnet gap 380 through the open end of the iron core 350. The one or more spiders 395 allow for relative movement between the voice coil 390 and the magnet 370 whilst minimizing or avoiding torsion and in-plane movements.

The voice coil transducer 300 is attached to the diaphragm 116. The attachment of the diaphragm 116 to a portion of the voice coil transducer 300 (such as the voice coil 390) may be by any suitable attachment means such as by adhesive. Alternatively, the diaphragm 116 and a portion of the voice coil 390 may be made as a single piece.

Additionally, the voice coil transducer 300 is attached to the frame 112 by the support members 118. Rotational movement of the frame 310 relative to the frame 112 is limited.

Movements induced in the acoustic waves will cause the diaphragm 116 to move, in turn inducing movement of the voice coil 390 within the magnet gap, resulting in an induced electrical signal.

In certain variations of the voice coil transducer, the configuration of the transducer is arranged to pick up more orthogonal signals than in-plane signals, thereby improving sensitivity. For example, the one or more spiders are designed to have out-of-plane compliance and be stiff in-plane. The same is true of the diaphragm 116 whose material and stiffness properties can be selected to improve out-of-plane compliance. The diaphragm may have a convex configuration (e.g. dome shaped) to further help in rejecting non-orthogonal signals by deflecting them away. Furthermore, signal processing may further derive any non-orthogonal signals e.g. by using a 3 axis accelerometer. This either to further reject non-orthogonal signals or even to particularly allow non-orthogonal signals through the sensor to derive the angle of origin of the incoming acoustic wave.

It will be appreciated that different uses of the sensing device may require different sensitivities and face different noise/signal ratios challenges. For example, higher sensitivity and increased signal/noise ratio will be required for clothing contact uses compared to direct skin contact uses. Similarly, higher sensitivity and increased signal/noise ratio will be required for non-contact uses compared to contact uses.

Therefore, in order to provide sensing devices having sensitivities and signal/noise ratios suitable for different form factors (e.g. contact or non-contact uses), developers have discovered that modulation of certain variables can optimize the voice coil transducer for the specific intended use: magnet strength, magnet volume, voice coil height, wire thickness, number of windings, number of winding layers, winding material (e.g. copper vs aluminum), and spider configuration. This is further explained in Example 1.

In certain variations, the voice coil 390 is configured to have an impedance of more than about 10 Ohms, more than about 20 Ohms, more than about 30 Ohms, more than about 40 Ohms, more than about 50 Ohms, more than about 60 Ohms, more than about 70 Ohms, more than about 80 Ohms, more than about 90 Ohms, more than about 100 Ohms, more than about 110 Ohms, more than about 120 Ohms, more than about 130 Ohms, more than about 150 Ohms, or about 150 Ohms. This is higher than a conventional heavy magnet voice coil transducer which has an impedance of about 4-8 Ohms. This is achieved by modulating one or more of the number of windings, wire diameter, and winding layers in the voice coil. Many permutations of these parameters are possible, and have been tested by the developers, as set out in Example 1. In one such variation, the voice coil comprises fine wire and was configured to have an impedance of about 150 Ohms, and associated lowered power requirement, by increasing the wire windings.

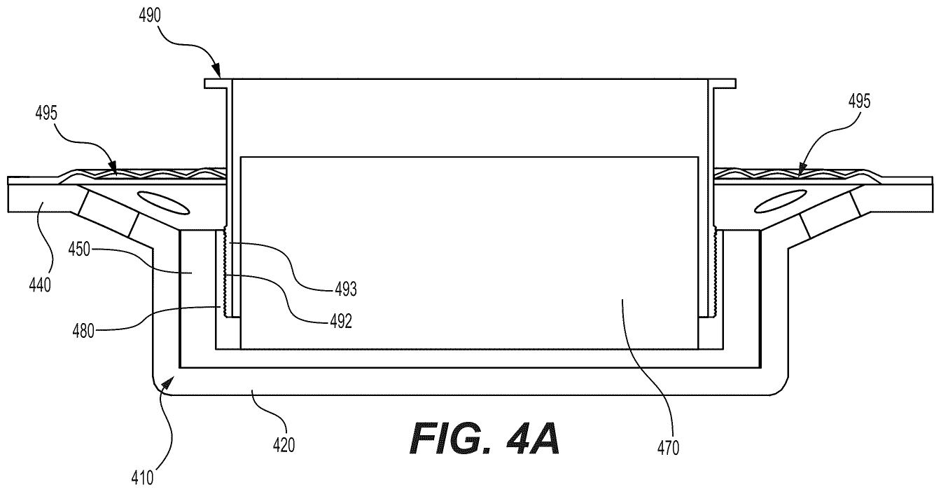

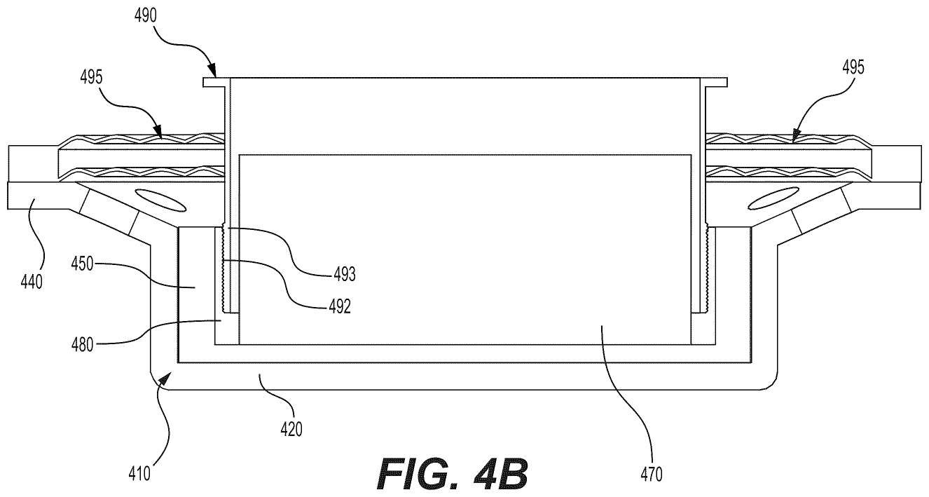

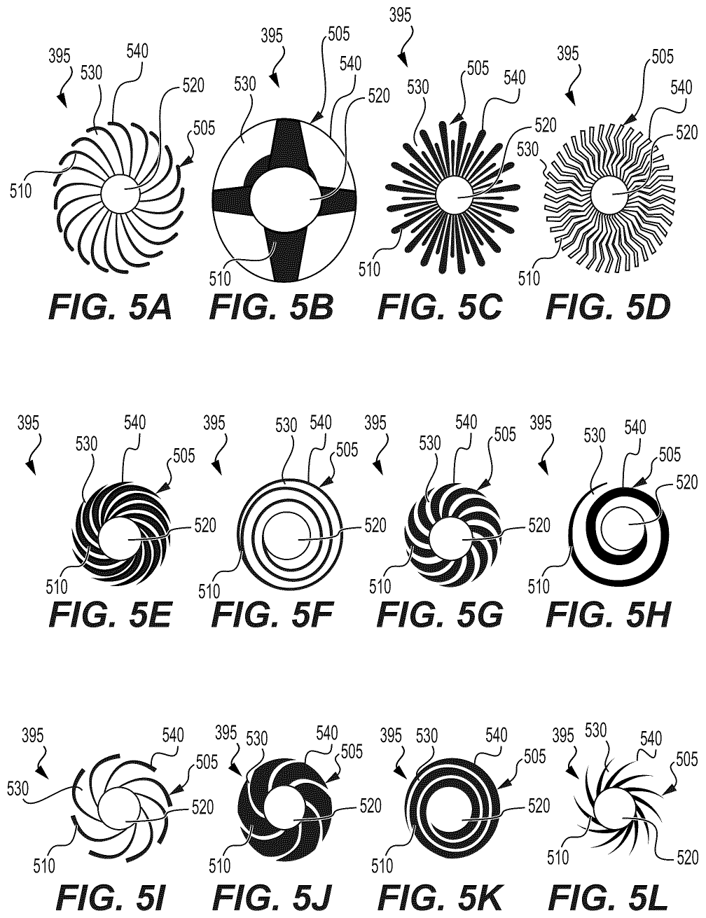

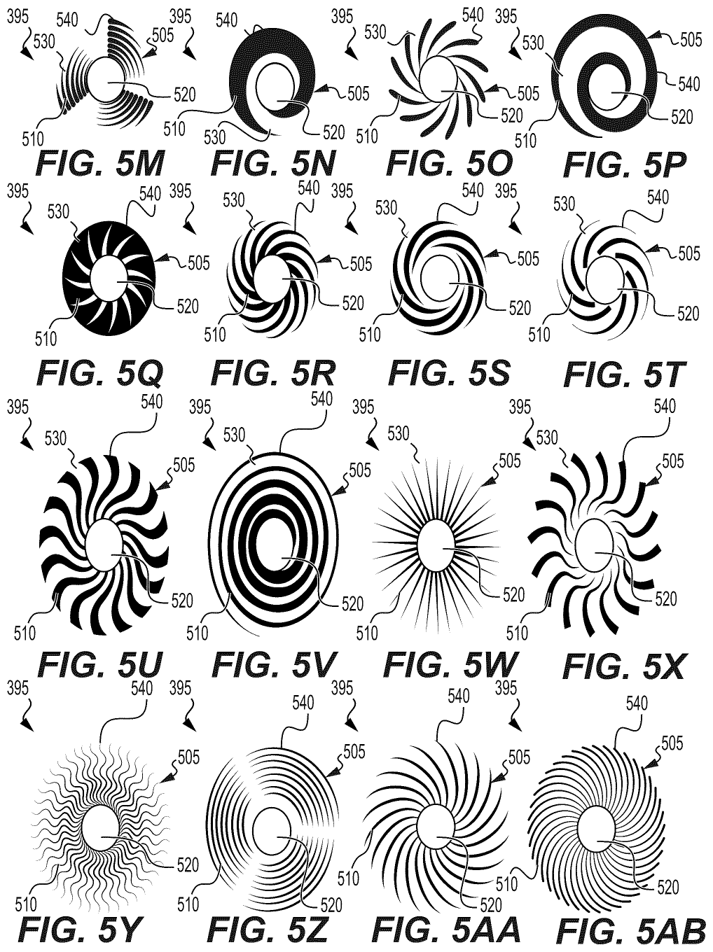

Developers also discovered that adaptation of the configuration of the spider 395 contributed to increasing sensitivity and signal/noise ratio increases. More specifically, it was determined via experiment and simulation that making the spider more compliant such as by incorporating apertures in the spider 395, increased sensitivity. Apertures also allow for free air flow. These are described in further detail below in relation to FIGS. 4A-4B and 5A-SAB. Alternatively, the spider 395 may be omitted and either the voice coil or the magnet is integrated into the diaphragm 116, whichever is of lower inertia due to mass and hence less restrictive in membrane movement.

The use of voice-coil based transducers for present uses is unintuitive, such as but not limited to contact with a body and/or the capture of sound below the audible threshold. Voice coils are commonly used in audio speaker systems and are optimized for the translation of electrical energy to acoustical energy. To achieve useful sound pressure levels, these audio speaker voice coils must be capable of handling high power in the range of 10 to 500 watts. The design considerations employed for this make them inappropriate for microphony or general sensing applications. Since electrical power can be described by the equation P=IV=V²/R, low resistance voice coils allow for high power handling at relatively low voltages, that are compatible with the power semiconductors typically used in audio amplifiers. In fact, most manufacturers of audio equipment note the ability of their amplifiers to drive low impedance speaker loads as advantages. While a high turn number, high impedance coil would be more efficient in terms of force generated for a unit current, the voltage required to drive such a current would require bulky insulation that would interfere with thermal management. While ferrofluid cooling is a possible solution, the viscosity of such fluids reduce sensitivity. Of course, when high power amplifiers are available, that is not an issue. Therefore, low impedance speakers, such as 8- and 4-Ohm models are relatively common. These are characterized by heavy voice coils and magnet structures built to accommodate the heavy windings that these coils comprise. Noise may also be a factor: temperature induced thermal noise increases with higher impedance of a conductor/resistor.

Moreover, in order to maintain reasonable efficiency at low frequencies of around 20 Hz, woofers and subwoofers typically use very heavy cones, so voice coil mass is not a critical issue. Contrary, tweeters need light voice coils to enable reasonable efficiency in air-diaphragm impedance matching using small diaphragms with higher frequency bandwidth, and are therefore very inefficient when operating at low frequencies. Tweeters typically have very light and delicate diaphragms as well, thus are not suitable for direct contact microphony.

Crossover circuitry is also usually necessary in order to achieve wide frequency response of audio speakers operating between 20 Hz and 20 kHz due to the need of two-way and three-way transducer speaker designs.

However, conventional microphones typically operate under totally different conditions, where low sound pressure levels need to be picked up with a minimum of noise. To such end, they are typically constructed with low weight diaphragms and the best microphones typically need external power sources as they operate as variable capacitors rather than as true voice coil/magnetic gap transducers. Again, just like tweeters, the delicate diaphragms of sensitive microphone designs are not suitable for direct contact microphony due to their fragility. Owing to their method of operation, they also suffer from low dynamic range and high natural resonance frequencies.

Therefore, the discovery that an adapted voice coil transducer can be used as a biosignal microphone was a surprising development by the Developers. In certain variations of the present technology, it was discovered that by adapting the configurations of at least the voice coil and the spider of a traditional heavy magnet structure audio speaker, it was possible to achieve a microphone with a higher sensitivity, broader frequency range detection capabilities, dynamic and tuneable frequency range, and high signal to noise characteristics. In certain variations, a single voice coil transducer of the current technology can provide a microphonic frequency response of less than about 1 Hz to over about 150 kHz or about 0.01 Hz to about 160 kHz.

Furthermore, the use of such a vibroacoustic sensor also enabled the size of the vibroacoustic sensor to be kept to a practical minimum for hand-held applications. These combinations of changes allowed for relatively higher voltage generation by the voice coil in response to vibroacoustic signals than would be possible using typical audio speaker voice coils. Consequently, the sensing of these voltages can be accomplished with low-noise J-FET based amplifiers, for example, to achieve the desired combination of frequency response, dynamic range, spurious signal rejection and signal to noise ratio.

In certain variations of the present technology, the voice coil transducer 300 comprises a single layer of spider 395 (FIG. 4A). In certain other variations of the present technology, the voice coil transducer 300 comprises a double layer of the spider 395 (FIG. 4B). Multiple spider 395 layers comprising three, four or five layers, without limitation, are also possible.

Certain configurations of the spider 395 are illustrated in FIGS. 5A-5AB. As can be seen, instead of a one-piece corrugated continuous configuration as is known in conventional spiders of conventional voice coils, in certain variations of the current technology, the spider 395 has a discontinuous surface. The spider 395 may comprise at least two deflecting structures 500 which are spaced from one another, permitting air flow therebetween. In certain configurations, the deflecting structures 500 comprises two or more arms 510 extending radially, and spaced from one another, from a central portion 520 of the spider 395. In the variation illustrated in FIGS. 4A and 4B, and 5B the deflecting structure 500 comprises four arms 519 extending radially from the central portion 520. The four arms 510 increase in width as they extend outwardly. Each of the arms 510 has a corrugated configuration. An aperture 530 between each of the arms 510 is larger than an area of each deflecting arm.

FIGS. 5A-5AB show other variants of the spider 395 for a voice coil transducer, such as the voice coil transducer 300. The spider 395 comprises one or more arms 510 extending from a central portion 520 and defining apertures 530 therebetween. The one or more arms 510 may be straight or curved. The one or more arms 510 may have a width which varies along its length, or which is constant along its length. The one or more arms 510 may be configured to extend from the central portion 520 in a spiral manner to a perimeter 540 of the spider 395. A solid ring may be provided at the perimeter 540 of the spider 395. This has been omitted from FIGS. 5A-5AB for clarity, but can be seen in FIG. 3E. In certain variations, there may be provided a single arm 510 configured to extend as a spiral from the central portion 520 of the spider 395 to the perimeter 540 of the spider 395. In these cases, turns of the spiral arms 510 define the apertures 530. The spider 395 may be defined as comprising a segmented form including portions that are solid (the arm(s) 510) and portions which are the aperture(s) 530 defined therebetween. The arms 510 may be the same or different (e.g. FIG. 5C). In variants where more than one layer of the spider 395 is provided in the voice coil transducer 300, the spiders 395 of each layer may be the same or different.

The configuration chosen for a given use of the sensing device 110 will depend on the amount of compliance required for that given use. For example, a voice coil configuration of high compliance may be chosen for the non-contact applications of the present technology.

In certain variations, a compliance of the diaphragm may range from about 0.4 to 3.2 mm/N. The compliance range may be described as low, medium and high, as follows: (i) 0.4 mm/N: low compliance->fs around 80-100 Hz; (ii) 1.3 mm/N: medium compliance->fs around 130 Hz; and (iii) 3.2 mm/N: high compliance->fs around 170 Hz.

In some variations, the sensing device 110 may include two or more voice coil transducers 300 which may enable triangulation of faint body sounds detected by the voice coil sensors, and/or to better enable cancellation and/or filtering of noise such as environmental disturbances. Sensor fusion data of two or more voice coil sensors can be used to produce low resolution sound intensity images.

In some variations, the voice coil transducer may be optimized for vibroacoustic detection, such as by using non-conventional voice coil materials and/or winding techniques. For example, in some variations, the voice coil material may include aluminum instead of conventional copper. Although aluminum has a lower specific conductance, overall sensitivity of the voice coil transducer may be improved with the use of aluminum due to the lower mass of aluminum. Additionally, or alternatively, the voice coil may include more than two layers or levels of winding (e.g., three, four, five, or more layers or levels), in order to improve sensitivity. In certain variants, the wire windings may comprise silver, gold or alloys for desired properties. Any suitable material may be used for the wire windings for the desired function. In certain other variants, the windings may be printed, using for example conductive inks onto the diaphragm.

The vibroacoustic sensor of certain variants of the present technology has advantages over conventional acoustic and electrical stethoscopes which are used to detect acoustic signals relating to the subject.

Firstly, the present technology can be deployed for contactless applications such as remote monitoring. On the other hand, traditional acoustic stethoscopes require contact with the skin of the subject for adequate sound detection.

Secondly, acoustic signals can be detected over a broad range and with good signal to noise ratios. Conversely, traditional acoustic stethoscopes have poor sound volume and clarity as they convert the movement of the stethoscope diaphragm into air pressure, which is directly transferred via tubing to the listener's ears by inefficient acoustic energy transfer. The listener therefore hears the direct vibration of the diaphragm via air tubes.

The current technology also has advantages over conventional electrical stethoscope transducers, which tend to be one of two types: (1) microphones mounted behind the stethoscope diaphragm, or (2) piezo-electric sensors mounted on, or physically connected to, the diaphragm.

Microphones mounted behind the stethoscope diaphragm pick up the sound pressure created by the stethoscope diaphragm, and convert it to electrical signals. The microphone itself has a diaphragm, and thus the acoustic transmission path comprises or consists of a stethoscope diaphragm, the air inside the stethoscope housing, and finally the microphone's diaphragm. The existence of two diaphragms, and the intervening air path, can result in excess ambient noise pickup by the microphone, as well as inefficient acoustic energy transfer. This inefficient acoustic energy transfer is a prevalent problem in the below-described electrical stethoscopes. Existing electronic stethoscopes use additional technologies to counteract this fundamentally inferior sensing technique, such as adaptive noise canceling and various mechanical isolation mountings for the microphone. However, these merely compensate for the inherent inadequacies of the acoustic-to-electrical transducers.

Piezo-electric sensors operate on a somewhat different principle than merely sensing diaphragm sound pressure. Piezo-electric sensors produce electrical energy by deformation of a crystal substance. In one case, the diaphragm motion deforms a piezoelectric sensor crystal mechanically coupled to the diaphragm, resulting in an electrical signal. The problem with this sensor is that the conversion mechanism can produce signal distortion compared with sensing the pure motion of the diaphragm. The resulting sound is thus somewhat different in tone, and distorted compared with an acoustic stethoscope.

Capacitive acoustic sensors are in common use in high-performance microphones and hydrophones. A capacitive microphone utilizes the variable capacitance produced by a vibrating capacitive plate to perform acoustic-to-electrical conversion. A capacitive microphone placed behind a stethoscope diaphragm would suffer from the same ambient noise and energy transfer problems that occur with any other microphone mounted behind a stethoscope diaphragm.

Acoustic-to-electrical transducers operate on a capacitance-to-electrical conversion principle detecting diaphragm movement directly, converting the diaphragm movement to an electrical signal which is a measure of the diaphragm motion. Further amplification or processing of the electrical signal facilitates the production of an amplified sound with characteristics very closely resembling the acoustic stethoscope sound, but with increased amplification, while maintaining low distortion.

This is a significant improvement over the more indirect diaphragm sound sensing produced by the microphonic or piezoelectric approaches described above. Since the diaphragm motion is sensed directly, the sensor is less sensitive to outside noise, and the signal is a more accurate measure of the diaphragm movement. With an acoustic stethoscope, diaphragm movement produces the acoustic pressure waves sensed by the listener's ears. With an acoustic-to-electrical sensor, that same diaphragm movement produces the electrical signal in a direct manner. The signal is used to drive an acoustic output transducer such as earphones or headphones, to set up the same acoustic pressure waves impinging on the listener's ears.

While acoustic-to-electrical transducers overcome many of the inherent problems faced by earlier stethoscope designs, it adds considerable white noise to the signal. White noise is a sound that contains every frequency within the range of human hearing (generally from 20 Hz to 20 kHz) in equal amounts. Most people perceive this sound as having more high-frequency content than low, but this is not the case. This perception occurs because each successive octave has twice as many frequencies as the one preceding it. For example, from 100 Hz to 200 Hz, there are one hundred discrete frequencies. In the next octave (from 200 Hz to 400 Hz), there are two hundred frequencies. As a result, the listener has difficulty discerning the human body sound from the white noise. For sounds of the body with higher intensities (i.e., louder sounds) the listener can hear the body sounds well, but lower-intensity sounds disappear into the background white noise. This is not the case in certain variations of the present technology.

1.h. Electric Potential Sensors

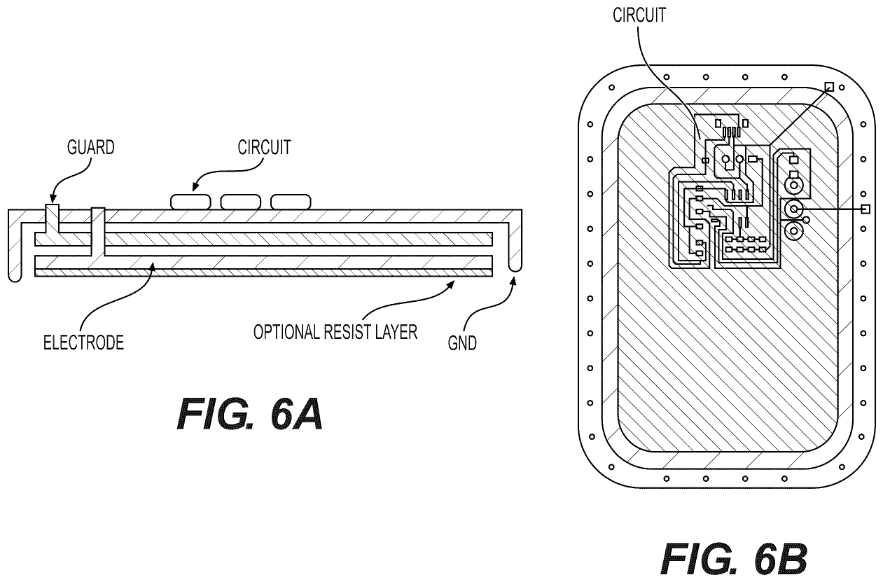

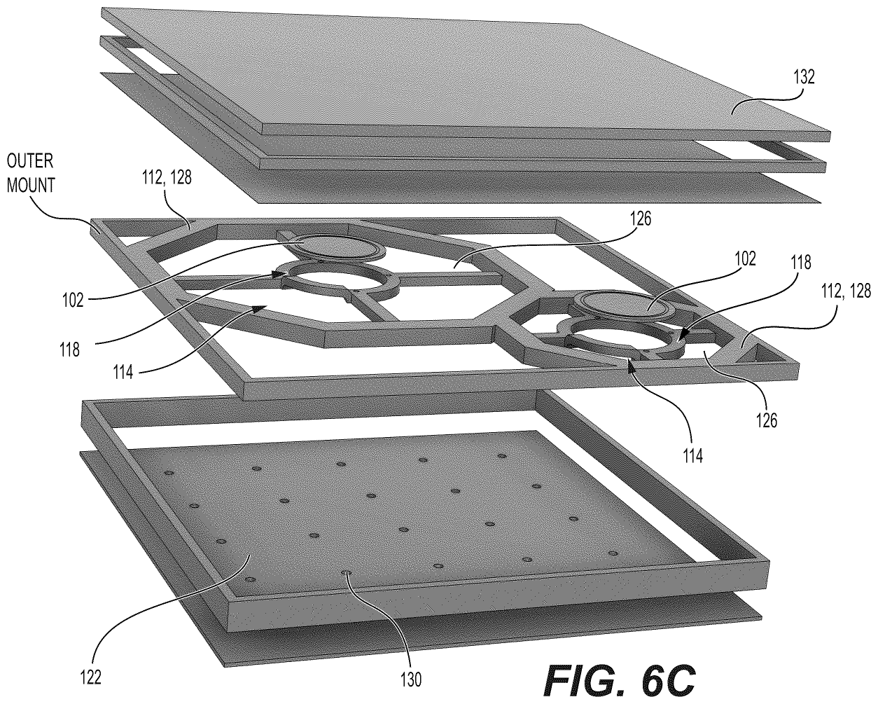

The sensor 101 used in the sensing device 110, in certain embodiments, comprises one or more Electric Potential Integrated Circuit (EPIC) sensors that allow non-contact, at a distance and through-clothing measurements. Certain EPIC sensors used within present systems and devices may include one or more as described in: U.S. Pat. Nos. 8,923,956; 8,860,401; 8,264,246; 8,264,247; 8,054,061; 7,885,700; the contents of which are herein incorporated by reference. A schematic diagram is shown in FIGS. 6A and 6B. The variation of the EPIC sensor illustrated in FIGS. 6A and 6B comprises layers of an electrode, a guard and a ground (GND). A circuit is positioned on top of the GND. The electrode may have an optional resist layer. FIG. 6C depicts an example EPIC sensor 102 in the sensing device 110 of FIG. 2A.

Electric Potential sensors (EPS) can pick up subtle movement of nearby objects due to the disturbance of static electric fields they cause. An EPS close to the diaphragm 116 is hence able to sense the motion of the vibrating diaphragm 116. In contrast to the voice coil-based sensor 101, the EPS sensor might not add any mass or additional spring constant and hence keeps the original compliance of the diaphragm 116 thereby avoiding a potential reduction in sensitivity.

In certain embodiments, the support member 112 is configured such that the aperture is defined in one of the first side 120 and the second side 124 only. The aperture 114 is covered by the diaphragm 116 on the one of the first side 120 and the second side 124. In certain embodiments, this may acoustically seal the cavity formed by the aperture 114. In other embodiments, a smaller opening may be provided on the other of the first side 120 and the second side 124 so that the cavity is not acoustically sealed.

In certain embodiments, the support member 112 is configured as a frame such that the aperture extends through the support member 112 between the first side 120 and the second side 124. In certain of these embodiments, the sensor 101 may be positioned in the cavity of the aperture 114 between the first side 120 and the second side. In certain others of these embodiments, the sensor 101 may be positioned outside of the cavity of the aperture 114, either on the first side 120 or the second side 124.

In certain embodiments, the diaphragm 116 may be provided with a layer configured to amplify electric potential pick-up for the EPIC sensor underneath. The layer may be a ferroelectric layer, which may be of nano- or micro thickness. Therefore, a weight added to the diaphragm 116 is minimal but it can enable a detection or improve a detection of diaphragm 116 vibrations depending on the material of the diaphragm 116. It will be appreciated that the EPIC sensor itself does not touch the diaphragm 116.

In certain embodiments, there may be provided one or more shields for minimizing or avoiding ingress of acoustic signals from given directions. For example, the outer mount, when present, could include a metal or another conductive material for grounding potential. In certain embodiments, a DRL may be provided to help to further reduce unwanted noise.

1.i. Capacitive Pickup Sensors

In certain embodiments, the sensor 101 is a capacitive microphone which is a direct alternative approach to the EPS pickup, however with the need of a layer on top of the diaphragm 116 with the ability to create a charge. A fixed metal plate is provided behind the diaphragm 116 in close proximity to complete the two components of a capacitor with the air gap in between acting as the dielectric. The metal plate could have any size from very small to the entire size of the diaphragm 116. Further, the layer on top of the diaphragm 116 can either be a conductive material that is polarized through an applied voltage (commonly known as Phantom Voltage) or could be an electret material that offers a quasi-permanent electric charge or dipole polarisation. In either case, the added layer adds mass to the vibrating membrane and hence inertia.

1.j. Magnetic Field Disturbance Sensors

In certain embodiments, the sensor 101 is a magnetic field disturbance sensor but without integration of any voice coil component within the membrane. The magnetic field of the sensor is routed through a ferromagnetic layer on the diaphragm 116. Diaphragm 116 vibrations modulate the magnetic field that hence induces a current in the voice coil winding resulting in a signal.

1.k. Photodetector and Light Source

In certain embodiments, the sensor 101 comprises a photodetector and light source positioned behind the diaphragm 116. The light source is positioned to direct an energy beam to the diaphragm 116 and a photodetector is positioned to detect the energy beam reflected from the diaphragm 116 and to measure a change in angle of the reflected energy beam (reflected of membrane movement). The reflection angle may depend on local bending of diaphragm 116, which in turn vibrates with incoming pressure waves. The photodetector may comprise a photodiode array from which the reflection angle is determined by the specific photodiode in the array, which captures the majority of the reflected signal intensity.

1.l. Strain Sensor

In certain embodiments, the sensor 101 comprises a strain sensor which can be positioned directly on the diaphragm 116 surface at strategic locations to detect movement of the diaphragm 116, e.g. layers of PVDF.

1.m. Acoustic Echo Dopplers

In certain embodiments, the sensor 101 comprises an acoustic echo doppler which can target a high frequency acoustic signal to the backside of the diaphragm 116, which is reflected into a detector. diaphragm 116 vibrations are frequency modulated into the Doppler carrier frequency, and demodulation results in a membrane vibration pickup. Acoustic Doppler could either operate in Pulsed Wave or Continuous Wave mode.

1.n. Echo Sensor-Based Vibroacoustic

Variants of the system 100 or the sensing device 110 may include one or more echo based sensors, such as but not limited to one or more of: echo sensors based on Continuous Wave Doppler (CWD), Pulsed Wave Doppler (PWD), and Time-of-Flight.

Continuous Wave Doppler (CWD): A continuous ultrasound signal is emitted by a source oscillator, reflected of a subject and back into a receiver. Vibrations on the subject change the frequency/phase of the emitted Ultrasound signal which allows to retrieve the original vibration signal. This offers maximum sampling frequency of the subject under investigation.

Pulsed Wave Doppler (PWD): Short ultrasound bursts are sent, and receiver waits for response. This technique can resolve subject vibrations like the CWD, but due to the burst interval introduces a sampling frequency of the subject. The Nyquist frequency of the corresponding sampling frequency is (pulses per second)/2. Hence with one pulse every millisecond the maximum resolved subject vibration frequency is 500 Hz. However, the PWD can resolve vibrations at a specific depth, or distance from the emitter/sensor. This is achieved by taking the time-of-flight information of the pulse into account and reject signals that outside the desired distance. Hence, the PWD can reject signals outside the target distance; signals that either are created by other sources or the emitted pulse that has traveled beyond the subject and reflected of a wall.

Time-of-Flight: A simpler version compared to the PWD is a pulsed ultrasound signal where only the time-of-flight is considered.

Advantageously, these echo-based sensors can permit measurement of vibrations (such as vibroacoustic signals from the subject), as well as distance or velocity. The echo-based sensors are non-contact, non-invasive and not harmful to the subject. Vibroacoustic signals can be detected from a distance of about 1 cm to about 10 meters, in certain variations. A detection distance may be about 10 meters, about 9 meters, about 8 meters, about 7 meters, about 6 meters, about 5 meters, about 4 meters, about 3 meters, about 2 meters or about 1 meter. Signal detection can be performed through clothing or other apparel of the subject. Furthermore, signal detection over a broad spectrum can be obtained.

The echo based acoustic systems broadly comprise an emitter component and a receiver component and are active systems which rely on the receiver component detecting a signal from the subject responsive to an emitted signal by the emitter incident on the subject. Therefore, in certain variations, emission signals within the ultrasound range are used, preferably above 25 kHz to keep some headroom to the end of the audible spectrum (as it is not desirable to use emission signals within the audible range). On the higher end, the maximum may be around 100 kHz due to ultrasonic signal absorption in air and ADC sampling rates. At 50 kHz the acoustic absorption in air is about 1-2 dB/m, at 100 kHz about 2-5 dB/m, at 500 kHz about 40-60 dB/m and at 1 Mhz about 150-200 dB/m. Technological challenges at higher frequencies involve the ability to capture the signal in sufficient quality, such as the availability of fast Analog-to-Digital converters.

The number of emitter components and receiver components in the echo sensor is not limited. Different combinations may be used as will be explained in further detail with reference to FIGS. 33D-H. For example, there may be provided a single emitter component and a single receiver component; or two emitter components and a single receiver component; or single emitter component and two receiver components; or two emitter components and two receiver components.

The emitter component can be any type of emitter configured to emit an ultrasound signal. Emitters should possibly be as unidirectional as possible. One example is the Pro-Wave Electronics 400ET/R250 Air Ultrasonic Ceramic Transducer.

The receiver component can be of any receiver type configured to detect the emitted ultrasound signal from the subject. In certain variations, the receiver component can be a microphone capable of capturing the ultrasound signal with sufficient signal-to-noise ratio. This could include any type of microphone such as condenser, dynamic or MEMS microphones. In certain variations, Ultrasound capable MEMS microphones are preferred due to compactness. In other variations, the receiver component is a specialized Ultrasound receiver that is tuned to that frequency. In certain variations, the receiver component is as unidirectional as possible. Examples of receiver components include the Pro-Wave Electronics 400ST/R100 Transducer; the Pro-Wave Electronics 400ST/R160 Transducer; or Invensense ICS-41352.

1.o. Laser Doppler Interferometers

In certain embodiments, the sensor 101 comprises a laser Doppler interferometer which utilizes the doppler effect and interference. In contrast to the Acoustic Echo Doppler this light-based approach results in higher SNR and amplitude resolution. Instead of using laser light, a setup includes a Time-of-Flight radar, radar doppler or any other commonly known radar sensing technology such as Ultra-Wideband-Radar (UWB). In addition to laser and radar, the vibration pickup could be based on any frequency of electromagnetic waves and combined with the same fundamental methodologies such as TOF and Doppler effect.

1.p. Additional Sensors in the System

The system 100 may comprise additional sensors, such as for detecting signals other than vibroacoustic signals associated with the subject or the environment, such as, without limitation, a contextual sensor, an echo doppler sensor, a kinetic sensor, temperature sensor, VOC sensor, machine vision sensor, an environmental a camera, a barometer, etc. for measuring one or more of ambient temperature, ambient humidity, ambient radiation; barometric pressure, altitude, ambient noise, and ambient light; IMU; GPS, a thermometer.

### Contextual Sensor

Little is known about what happens in real life, how lifestyle and daily context impacts vital signs, how quality of life is impacted by disease and medical conditions and to what degree therapeutic and care recommendations are actually adhered to. Developers have determined that putting health data and care into context of daily life, can in certain variations, add key insights to get richer and personalized interpretation of biosignals, vital signs, and wellbeing. In some variations, the system 100 may further include one or more sensors providing environmental and/or other contextual data (e.g., social determinants of health). This may be used to calibrate and/or better interpret the vibroacoustic data acquired with the vibroacoustic sensor, or any of the other sensors. Such data (e.g., environmental and/or social determinants of health) may, for example, help contextualize data for more accurate machine learning and/or AI data analysis. For example, in some variations, the sensing device 110 or the system 100 may include a contextual sensor. The contextual sensor may be in communication with the processors 105 in the computing system 102 or in the electronics system of the sensing device 110 such that sensor data from the contextual sensor may be taken into account when analyzing vibroacoustic data and/or other suitable data.

The contextual sensor may include one or more suitable sensors such as environmental sensors to measure one or more ambient characteristics and/or one or more characteristics of the sensing device relative to the environment. For example, the contextual sensor may include an ambient light sensor, an ambient noise sensor (microphone), an ambient humidity sensor, an ambient pressure sensor, an ambient temperature sensor, an air quality sensor (e.g., detection of volatile organic compounds (VOCs)), altitude sensor (e.g., relative pressure sensor), GPS, and/or other suitable sensor(s) to characterize the environment in which the sensing device is operating. Additionally, or alternatively, the contextual sensor may include an inertial measurement unit (IMU), individual gyroscope and/or accelerometer, and/or other suitable sensor(s) to characterize the sensing device relative to the environment.

These may be useful for contextualizing the relevant sensor data collected. Additionally, or alternatively, ambient environmental data (e.g., ambient noise) may be used for noise cancellation from the relevant biological vibroacoustic signal component. Such noise cancellation may, for example, be performed as active noise cancellation on the device, or as a postprocessing step.

### Acoustocardiography (ACG) Sensor

In some variations, the system 100 may further include one or more sensor for detecting vibrations of the heart as the blood moves through the various chambers, valves, and large vessels, using an acoustic cardiography sensor. The ACG sensor can record these vibrations at four locations of the heart and provides a “graph signature.” While the opening and closing of the heart valves contributes to the graph, so does the contraction and strength of the heart muscle. As a result, a dynamic picture is presented of the heart in motion. If the heart is efficient and without stress, the graph is smooth and clear. If the heart is inefficient, there are definite patterns associated each type of contributing dysfunction. The ACG is not the same as an ECG, which is a common diagnostic test. The electrocardiograph (ECG) records the electrical impulses as it moves through the nerves of the heart tissue as they appear on the skin. The ECG primarily indicates if the nervous tissue network of the heart is affected by any trauma, damage (for example from a prior heart attack or infection), severe nutritional imbalances, stress from excessive pressure. Only the effect on the nervous system is detected. It will not tell how well the muscle or valves are functioning, etc. In addition, the ECG is primarily used to diagnose a disease. The ACG not only looks at electrical function but also looks at heart muscle function, which serves as a window of the metabolism of the entire nervous system and the muscles. Using the heart allows a “real-time” look at the nerves and muscles working together. As a result of this interface, unique and objective insights into health of the heart and the entire person can better be seen.

### Passive Acoustocerebrography (ACG) Sensor

In some variations, the system 100 may further include one or more passive acoustocerebrography sensor for detecting blood circulation in brain tissue. This blood circulation is influenced by blood circulating in the brain's vascular system. With each heartbeat, blood circulates in the skull, following a recurring pattern according to the oscillation produced. This oscillation's effect, in turn, depends on the brain's size, form, structure and its vascular system. Thus, every heartbeat stimulates minuscule motion in the brain tissue as well as cerebrospinal fluid and therefore produces small changes in intracranial pressure. These changes can be monitored and measured in the skull. The one or more passive acoustocerebrography sensors may include passive sensors like accelerometers to identify these signals correctly. Sometimes highly sensitive microphones can be used.

### Active Acoustocerebrography (ACG) Sensor

In some variations, the system 100 may further include one or more active acoustocerebrography sensors. Active ACG sensors can be used to detect a multi-frequency ultrasonic signal for classifying adverse changes at the cellular or molecular level. In addition to all of the advantages that passive ACG sensors provide, the active ACG sensor can also conduct a spectral analysis of the acoustic signals received. These spectrum analyses not only display changes in the brain's vascular system, but also those in its cellular and molecular structures. The active ACG sensor can also be used to perform a Transcranial Doppler test, and optionally in color. These ultrasonic procedures can measure blood flow velocity within the brain's blood vessels. They can diagnose embolisms, stenoses and vascular constrictions, for example, in the aftermath of a subarachnoid hemorrhage.

### Ballistocardiography (BCG) Sensor

In some variations, the system 100 may further include one or more ballistocardiograph sensors (BCG) for detecting ballistic forces generated by the heart. The downward movement of blood through the descending aorta produces an upward recoil, moving the body upward with each heartbeat. As different parts of the aorta expand and contract, the body continues to move downward and upward in a repeating pattern. Ballistocardiography is a technique for producing a graphical representation of repetitive motions of the human body arising from the sudden ejection of blood into the great vessels with each heart beat. It is a vital sign in the 1-20 Hz frequency range which is caused by the mechanical movement of the heart and can be recorded by noninvasive methods from the surface of the body. Main heart malfunctions can be identified by observing and analyzing the BCG signal. BCG can also be monitored using a camera-based system in a non-contact manner. One example of the use of a BCG is a ballistocardiographic scale, which measures the recoil of the person's body who is on the scale. A BCG scale is able to show a person's heart rate as well as their weight.

### Electromyography (EMG) Sensor

In some variations, the system 100 may further include one or more Electromyography (EMG) sensors for detecting electrical activity produced by skeletal muscles. The EMG sensor may include an electromyograph to produce a record called an electromyogram. An electromyograph detects the electric potential generated by muscle cells when these cells are electrically or neurologically activated. The signals can be analyzed to detect medical abnormalities, activation level, or recruitment order, or to analyze the biomechanics of human or animal movement. EMG can also be used in gesture recognition.

### Electrooculography (EOG) Sensor

In some variations, the system 100 may further include one or more electrooculography (EOG) sensors for measuring the corneo-retinal standing potential that exists between the front and the back of the human eye. The resulting signal is called the electrooculogram. Primary applications are in ophthalmological diagnosis and in recording eye movements. Unlike the electroretinogram, the EOG does not measure response to individual visual stimuli. To measure eye movement, pairs of electrodes are typically placed either above and below the eye or to the left and right of the eye. If the eye moves from center position toward one of the two electrodes, this electrode “sees” the positive side of the retina and the opposite electrode “sees” the negative side of the retina. Consequently, a potential difference occurs between the electrodes. Assuming that the resting potential is constant, the recorded potential is a measure of the eye's position.

### Electroolfactography (EOG) Sensor

In some variations, the system 100 may further include one or more Electro-olfactography or electroolfactography (EOG) sensors for detecting a sense of smell of the subject. The EOG sensor can detect changing electrical potentials of the olfactory epithelium, in a way similar to how other forms of electrography (such as ECG, EEG, and EMG) measure and record other bioelectric activity. Electro-olfactography is closely related to electroantennography, the electrography of insect antennae olfaction.

### Electroencephalography (EEG) Sensor

In some variations, the system 100 may further include one or more electroencephalography (EEG) sensors for electrophysiological detection of electrical activity of the brain, or vibroacoustic sensors placed onto the skull anechoic chamber to “listen” to the brain and capture subtle pressure and pressure gradient changes related to the speech processing circuitry. EEG is typically noninvasive, with the electrodes placed along the scalp, although invasive electrodes are sometimes used, as in electrocorticography. EEG measures voltage fluctuations resulting from ionic current within the neurons of the brain. Clinically, EEG refers to the recording of the brain's spontaneous electrical activity over a period of time, as recorded from multiple electrodes placed on the scalp. Diagnostic applications generally focus either on event-related potentials or on the spectral content of EEG. The former investigates potential fluctuations time locked to an event, such as ‘stimulus onset’ or ‘button press’. The latter analyses the type of neural oscillations (popularly called “brain waves”) that can be observed in EEG signals in the frequency domain. EEG can be used to diagnose epilepsy, which causes abnormalities in EEG readings. It can also used to diagnose sleep disorders, depth of anesthesia, coma, encephalopathies, and brain death. EEG, as well as magnetic resonance imaging (MRI) and computed tomography (CT) can be used to diagnose tumors, stroke and other focal brain disorders. Advantageously, EEG is a mobile technique available and offers millisecond-range temporal resolution which is not possible with CT, PET or MRI. Derivatives of the EEG technique include evoked potentials (EP), which involves averaging the EEG activity time-locked to the presentation of a stimulus of some sort (visual, somatosensory, or auditory). Event-related potentials (ERPs) refer to averaged EEG responses that are time-locked to more complex processing of stimuli.

### Ultra-Wideband (UWB) Sensor

In some variations, the system 100 may further include one or more ultra-wideband sensors (also known as UWB, ultra-wide band and ultraband). UWB is a radio technology that can use a very low energy level for short-range, high-bandwidth communications over a large portion of the radio spectrum. UWB has traditional applications in non-cooperative radar imaging. Most recent applications target sensor data collection, precision locating and tracking applications. A significant difference between conventional radio transmissions and UWB is that conventional systems transmit information by varying the power level, frequency, and/or phase of a sinusoidal wave. UWB transmissions transmit information by generating radio energy at specific time intervals and occupying a large bandwidth, thus enabling pulse-position or time modulation. The information can also be modulated on UWB signals (pulses) by encoding the polarity of the pulse, its amplitude and/or by using orthogonal pulses. UWB pulses can be sent sporadically at relatively low pulse rates to support time or position modulation, but can also be sent at rates up to the inverse of the UWB pulse bandwidth. Pulse-UWB systems have been demonstrated at channel pulse rates in excess of 1.3 gigapulses per second using a continuous stream of UWB pulses (Continuous Pulse UWB or C-UWB), supporting forward error correction encoded data rates in excess of 675 Mbit/s.

A valuable aspect of UWB technology is the ability for a UWB radio system to determine the “time of flight” of the transmission at various frequencies. This helps overcome multipath propagation, as at least some of the frequencies have a line-of-sight trajectory. With a cooperative symmetric two-way metering technique, distances can be measured to high resolution and accuracy by compensating for local clock drift and stochastic inaccuracy.

Another feature of pulse-based UWB is that the pulses are very short (less than 60 cm for a 500 MHz-wide pulse, and less than 23 cm for a 1.3 GHz-bandwidth pulse)—so most signal reflections do not overlap the original pulse, and there is no multipath fading of narrowband signals. However, there is still multipath propagation and inter-pulse interference to fast-pulse systems, which must be mitigated by coding techniques.

Ultra-wideband is also used in “see-through-the-wall” precision radar-imaging technology, precision locating and tracking (using distance measurements between radios), and precision time-of-arrival-based localization approaches. It is efficient, with a spatial capacity of about 1013 bit/s/m². UWB radar has been proposed as the active sensor component in an Automatic Target Recognition application, designed to detect humans or objects that have fallen onto subway tracks.

Ultra-wideband pulse Doppler radars can also be used to monitor vital signs of the human body, such as heart rate and respiration signals as well as human gait analysis and fall detection. Advantageously, UWB has less power consumption and a high-resolution range profile compared to continuous-wave radar systems.

### Seismocardiography (SCG) Sensor

In some variations, the system 100 may further include one or more seismocardiography (SCG) sensor for non-invasive measurement of cardiac vibrations transmitted to the chest wall by the heart during its movement. SCG can be used to assess the timing of different events in the cardiac cycle. Using these events, assessing, for example, myocardial contractility might be possible. SCG can also be used to provide enough information to compute heart rate variability estimates. A more complex application of cardiac cycle timings and SCG waveform amplitudes is the computing of respiratory information from the SCG.

### Intracardiac Electrogram (EGM) Sensor

In some variations, the system 100 may further include one or more intracardiac electrogram (EGM) sensors for non-invasive measurement of cardiac electrical activity generated by the heart during its movement. It provides a record of changes in the electric potentials of specific cardiac loci as measured by electrodes placed within the heart via cardiac catheters; it is used for loci that cannot be assessed by body surface electrodes, such as the bundle of His or other regions within the cardiac conducting system.

### Pulse Plethysmograph (PPG) Sensor

In some variations, the system 100 may further include one or more pulse plethysmograph (PPG) sensors for non-invasive measurement of the dynamics of blood vessel engorgement. The sensor may use a single wavelength of light, or multiple wavelengths of light, including far infrared, near infrared, visible or UV. For UV light, the wavelengths used are between about 315 nm and 400 nm and the sensor is intended to deliver less than 8 milliwatt-hours per square centimeter per day to the subject during its operation.

### Galvanic Skin Response (GSR) Sensor

In some variations, the system 100 may further include one or galvanic skin response (GSR) sensors. These sensors may utilize either wet (gel), dry, or non-contact electrodes as described herein.

### Volatile Organic Compounds (VOC) Sensor

In some variations, the system 100 may further include one or more volatile organic compounds (VOC) sensors for detecting VOC or semi-VOCs in exhaled breath of the subject. Exhaled breath analysis can permit the diagnosis and monitoring of disease. Certain VOCs are linked to biological processes in the human body. For instance, dimethylsulfide is exhaled as a result of fetor hepaticus and acetone is excreted via the lungs during ketoacidosis in diabetes. Typically, VOC Excretion or Semi-VOC excretion can be measured using plasmon surface resonance, mass spectroscopy, enzymatic based, semiconductor based or imprinted polymer-based detectors.

### Vocal Tone Inflection (VTI) Sensor

In some variations, the system 100 may further include one or more vocal tone inflection (VTI) sensors modules. VTI analysis can be indicative of an array of mental and physical conditions that make the subject slur words, elongate sounds, or speak in a more nasal tone. They may even make the subject's voice creak or jitter so briefly that it's not detectable to the human ear. Furthermore, vocal tone changes can also be indicative of upper or lower respiratory conditions, as well as cardiovascular conditions. Developers have found that VTI analysis can be used for early diagnosis of certain respiratory conditions from a Covid-19 infection.

### Capacitive Sensor

In some variations, the system 100 may further include one or more capacitive/non-contact sensors. Such sensors may include non-contact electrodes. These electrodes were developed since the absence of impedance adaptation substances could make the skin-electrode contact instable over time. This difficulty was addressed by avoiding physical contact with the scalp through non-conductive materials (i.e., a small dielectric between the skin and the electrode itself): despite the extraordinary increase of electrode impedance (>200 MOhm), in this way it will be quantifiable and stable over time.

A particular type of dry electrode, is known as a capacitive or insulated electrode. These electrodes require no ohmic contact with the body since it acts as a simple capacitor placed in series with the skin, so that the signal is capacitively coupled. The received signal can be connected to an operational amplifier and then to standard instrumentation.

The use of a dielectric material in good contact to the skin results in a fairly large coupling capacitance, ranging from 300 pF to several nano-farads. As a result, a system with reduced noise and appropriate frequency response is readily achievable using standard high-impedance FET (field-effect transistor) amplifiers.

While wet and dry electrodes require physical contact with the skin to function, capacitive electrodes can be used without contact, through an insulating layer such as hair, clothing or air. These contactless electrodes have been described generally as simple capacitive electrodes, but in reality there is also a small resistive element, since the insulation also has a non-negligible resistance.

The capacitive sensors can be used to measure heart signals, such as heart rate, in subjects via either direct skin contact or through one and two layers of clothing with no dielectric gel and no grounding electrode, and to monitor respiratory rate. High impedance electric potential sensors can also be used to measure breathing and heart signals.

### Capacitive Plates Sensor

In some variations, the system 100 may further include one or more capacitive plate sensors. Surprisingly, Developers discovered that the resistive properties of the human body may also be interrogated using the changes in dielectric properties of the human body that come with difference in hydration, electrolyte, and perspiration levels. In this variation, the sensing device may comprise two parallel capacitive plates which are positionable on either side of the body or body part to be interrogated. A specific time varying potential is applied to the plates, and the instantaneous current required to maintain the specific potential is measured and used as input into the machine learning system to correlate the physiological states to the data. As the dielectric properties of the body or body part changes with resistance, the changes are reflected in the current required to maintain the potential profile. In certain variations, a target bodily condition can be screened using such a capacitive plate and permitting interrogation of the subject standing on the capacitive plate.

### Machine Vision Sensor

In some variations, the system 100 may further include one or more machine vision sensors comprising one or more optical sensors such as cameras for capturing the motion of the subject, or parts of the subject, as they stand or move (e.g. walking, running, playing a sport, balancing etc.). In this manner, physiological states that affect kinesthetic movements such as balance and gait patterns, tremors, swaying or favoring a body part can be detected and correlated with the other data obtained from the other sensors in the apparatus such as center of mass positioning. Machine vision allows skin motion amplification to accurately measure physiological parameters such as blood pressure, heart rate, and respiratory rate. For example, heart/breath rate, heart/breath rate variability, and lengths of heart/breath beats can be estimated from measurements of subtle head motions caused in reaction to blood being pumped into the head, from hemoglobin information via observed skin color, and from periodicities observed in the light reflected from skin close to the arteries or facial regions. Aspects of pulmonary health can be assessed from movement patterns of chest, nostrils and ribs.

A wide range of motion analysis systems allow movement to be captured in a variety of settings, which can broadly be categorized into direct (devices affixed to the body, e.g. accelerometry) and indirect (vision-based, e.g. video or optoelectronic) techniques. Direct methods allow kinematic information to be captured in diverse environments. For example, inertial sensors have been used as tools to provide insight into the execution of various movements (walking gait, discus, dressage and swimming) Sensor drift, which influences the accuracy of inertial sensor data, can be reduced during processing; however, this is yet to be fully resolved and capture periods remain limited. Additionally, it has been recognized that motion analysis systems for biomechanical applications should fulfil the following criteria: they should be capable of collecting accurate kinematic information, ideally in a timely manner, without encumbering the performer or influencing their natural movement. As such, indirect techniques can be distinguished as more appropriate in many settings compared with direct methods, as data are captured remotely from the participant imparting minimal interference to their movement. Indirect methods were also the only possible approach for biomechanical analyses previously conducted during sports competition. Over the past few decades, the indirect, vision-based methods available to biomechanists have dramatically progressed towards more accurate, automated systems. However, there is yet to be a tool developed which entirely satisfies the aforementioned important attributes of motion analysis systems. Thus, these analyses may be used in coaching and physical therapy in dancing, running, tennis, golf, archery, shooting biomechanics and other sporting and physical activities. Other uses include ergonomic training for occupations that subject persons to the dangers of repetitive stress disorders and other physical stressors related to motion and posture. The data can also be used in the design of furniture, self-training, tools, and equipment design.

The machine vision sensor may include one or more digital camera sensors for imaging one or more of pupil dilation, scleral erythema, changes in skin color, flushing, and/or erratic movements of a subject, for example. Other optical sensors may be used that operate with coherent light, or use a time of flight operation. In certain variants, the machine vision sensor comprises a 3D camera such Astra Embedded S by Orrbec.

### Thermal Sensor

In some variations, the system 100 may further include one or more thermal sensors including an infrared sensor, a thermometer, or the like. The thermal sensor may be incorporated with the sensing device 110 or be separate thereto. The thermal sensor may be used to perform temperature measurements of one or more of a lacrimal lake and/or an exterior of tear ducts of the subject. In some variations, the thermal sensor may comprise a thermopile on a gimbal, such as but not limited to a thermopile comprising an integrated infrared thermometer, 3V, single sensor (not array), gradient compensated, medical+−0.2 to +−0.3 degree kelvin/Centigrade, 5 degree viewing angle (Field of view—FOV)

### Strain Gage Sensor

In some variations, the system 100 may comprise one or more strain gauge sensors that may be used to measure the subject's weight. In other variations these sensors may be used to acquire seismocardiograms or ballistocardiograms. These sensors, without limitations, may be resistive or piezo-electric strain gauges.

### Sensor Combinations

Any combination of the abovementioned sensor 101 and one or more additional sensors can be used in variants of the present system 100. The sensor combinations may be housed within the sensing device 110 or across multiple devices.

1.p. Electronics System

In some variations, the sensing device 110 may further include an electronics system. The electronics system may include various electronics components for supporting operation of the sensor 101 and the sensing device 110. For example, at least a portion of the electronics system may include a circuit board arranged in the support member 112. The electronics system may be configured to perform signal conditioning, data analysis, power management, communication, and/or other suitable functionalities of the device. The electronics system may be in communication with other components of the system 100 such as the computing system 102 and the processor 105. The electronics system may, in some variations, function as a microcontroller unit module for the sensing device 110 and may therefore include at least one processor, at least one memory device, suitable signal processing circuitry, at least one communication module for communicating with the computer system 102, and/or at least one power management module managing a power supply. One or more of these components or modules may be arranged one or more electronic circuit boards (e.g., PCB) which in turn may be mounted relative to the support member 112. In some variations, the electronics system may also include a microphone and/or speaker for enabling further functionality such as voice or data recording (e.g., permitting recitation of medical notes for an electronic health record, etc.).

1.q. Computer System

The processor 105 (e.g., CPU) and/or memory device (which can include one or more computer-readable storage mediums) may cooperate to provide a controller for operating the system 100. For example, the processor 105 may be configured to set and/or adjust sampling frequency for any of the various sensors 101 in the system 100. As another example, the processor 105 may receive sensor data (e.g., before and/or or after sensor signal conditions) and the sensor data may be stored in one or more memory devices. In some variations, some or all of the data stored on the memory device may be encrypted using a suitable encryption protocol (e.g., for HIPAA-compliant security). In some variations, the processor 105 and memory device may be implemented on a single chip, while in other variations they may be implemented on separate chips.

The computing system 102 may include a communication module configured to communicate data to one or more networked devices, such as a hub paired with the system 100, a server, a cloud network, etc. In some variations, the communication module may be configured to communicate information in an encrypted manner. While in some variations the communication module may be separate from the processor 105 as a separate device, in variations at least a portion of the communication module may be integrated with the processor (e.g., the processor may include encryption hardware, such as advanced encryption standard (AES) hardware accelerator (e.g., 128/256-bit key) or HASH (e.g., SHA-256)).

The communication module of the sensing device 110 or of the computing system 102 may communicate via a wired connection (e.g., including a physical connection such as a cable with a suitable connection interface such as USB, mini-USB, etc.) and/or a wireless network (e.g., through NFC, Bluetooth, WiFi, RFID, or any type of digital network that is not connected by cables). For example, devices may directly communicate with each other in pairwise connection (1:1 relationship), or in a hub-spoke or broadcasting connection (“one to many” or 1:m relationship). As another example, the devices may communicate with each other through mesh networking connections (e.g., “many to many”, or m:m relationships), such as through Bluetooth mesh networking. Wireless communication may use any of a plurality of communication standards, protocols, and technologies, including but not limited to, Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (WiFi) (e.g., IEEE 802.11a, IEEE 802.11b, IEEE 802.11g, IEEE 802.11n, and the like), or any other suitable communication protocol. Some wireless network deployments may combine networks from multiple cellular networks (e.g., 3G, 4G, 5G) and/or use a mix of cellular, WiFi, and satellite communication.

In some variations, the communication module may include multiple data communication streams or channels to help ensure broad spectrum data transfer (e.g., Opus 20 kHz with minimal delay codec). Such multiple data communication streams are an improvement over typical wireless data transmission codecs. For example, most wireless data transmission codecs (e.g., G.711) use a bandpass filter to only encode the optimal range of human speech, 300 Hz to 3,400 Hz (this is commonly referred to as a narrowband codec). As another example, some wireless data transmission codecs (e.g., G.722) encodes the range from 300 Hz to 7,000 Hz (this is commonly referred to as a wideband codec). However, most of the energy is concentrated below 1,000 Hz and there is virtually no audible sound above 5,000 Hz, while there is a measurable amount of energy above the 3,400 Hz cutoff of most codecs. The data throughput requirements for both G.711 and G.722 are the same because the modulation used in G.722 is a modified version of the PCM called Adaptive Differential Pulse Code Modulation (ADPCM). When this kind of complexity is added to a codec and process power remains constant, this will add latency. As such, G.711 will introduce latency well below just one millisecond but G.722 could introduce tens of milliseconds of delay—which is an unacceptably long delay in vibroacoustics.

### Sensor Data

In certain embodiments, the computing system 102 of the system 100 and/or the processor of the sensing device 110 may be configured to control sensor data acquisition and sensor data processing. For example, the sensor data may be captured as catenated raw amplitude sequences or as combined short-time Fourier transform spectra. In certain variations, the sensor data is captured in less than 15 seconds per subject, and preferably in less than 10 seconds per subject. In certain embodiments, the sensor data is acquired in data segments of about 15 s to about 20 s in length, or about 10 to about 25 s, or any other data segment length which satisfies data quality and data quantity requirements. In certain embodiments, sensor data is collected about 2 days to about 4 days for continuous health characterization and baselining.

In certain embodiments, the sensor data is collected and/or monitored in one or both of a baseline phase and a base-line update phase. This may correct for physiological drift. In the baseline phase, sensor data may be collected and/or monitored over 1 to 5 days, 1 to 4 days, 1 to 3 days, 1 to 2 days, 2 to 5 days, 3 to 5 days, 4 to 5 days, 1 to 3 days, 2 to 3 days. Data collection may be continuous or in data segments. In the update phase, biometric data may be collected and/or monitored for 1 to 25 seconds, 5 to 25 seconds, 10 to 25 seconds, 15 to 25 seconds, 20 to 25 seconds, 1 to 20 seconds, 1 to 15 seconds, 1 to 10 seconds, 5 to 10 seconds, 5 to 15 seconds. In certain embodiments, updates using baselined data requires a shorter confirmatory data read or top-up from about 5 seconds to about 10 seconds.

In certain embodiments, the method comprises acquiring biometric data of a subject at a first point in time, and storing in a database (“pre-screening step). The method further comprises, at the second point in time, acquiring the sensor data and using the stored data for monitoring or diagnosis. The pre-screening process may be carried out over a period of about 1 to 5 days. In certain embodiments, the baseline data is ephemeral (can be deleted, over written, or loses validity).

In certain embodiments, methods of the present technology may comprise collecting and/or monitoring the data with sampling rates from about 0.01 Hz to about 20 THz, more than about 10 THz, about 10 THz to about 100 THz; about 0.01 Hz to about 100 THz. In certain embodiments, this can result in improved data quality and quality meaning. These sampling rates may be considered as “high resolution” compared to conventional data sampling. In the Terahertz range, the date may be passively or actively captured.

1.r. Signal Processing System

Various analog and digital processes may process the sensor data for extracting useful signal from noise and communicate suitable data to one or more external host devices (e.g., computing device such as mobile device, one or more storage devices, medical equipment, etc.). At least a portion of the signal processing chain may occur in the sensing device 110 or the processor 105.

In some variations, a signal processing chain for handling data, such as the sensor data, ECG data, the contextual data, thermal data, optical data, etc. may be configured to provide an output signal with low noise (high signal-to-noise ratio (SNR), provide sufficient amplification to allow proper digitization of the analog signal, and function in a manner that keeps the overall signal fidelity sufficiently high. The signal processing chain may also be configured to (i) overcome signal attenuation and loss of strength of a signal as it propagates over a medium or a plurality of media, and/or (ii) to move digitized data sufficiently quickly through the various components of the sensing device to as to avoid significant signal and/or data loss. In some variations, the signal processing chain may include a programmable gain stage to adjust gain in real-time during operation of the sensing device in order to optimize signal range for analog-to-digital converters. The frequency and bandwidth requirements of the signal processing chain may vary depending specific applications, but in some variations the signal processing chain may have a sufficient bandwidth to sample frequencies up to about 160 kHz or up to about 320 kHz, and have a low frequency response of about 0.1 Hz or lower.

High-precision signal control may be important in biofield and other vibroacoustic active and passive sensing to minimize signal and/or data loss. However, the difficulty to obtain model parameters is one of the main obstacles to obtain high-precision tracking control of biofield signals using a model-dependent method. The vibroacoustic system with uncertain parameters can defend against signal and/or data loss by having high precision of the system output information. In some variations, an adaptive output feedback control scheme may be implemented with an inline servo system with uncertain parameters and unmeasurable states instantiated with controller and parameter adaptation algorithms to guarantee that the biofield signal tracking error is uniformly bounded. This method may be combined with a traditional proportional-integral-derivative (PID) control method with optimal parameters (e.g., obtained using a genetic algorithm), a sliding mode control based on exponential reaching law, and/or adaptive control methods and adaptive backstepping sliding mode control, to achieve higher tracking accuracy. The vibroacoustic system also may have better anti-interference ability with respect to signal load change.

Vibroacoustic signal control, which may be termed active vibro-acoustic control, can be achieved in some variations with multiple servo motors, actuators and sensors and fully-coupled feedforward or feedback controllers. For example, in some variations, feedback may be achieved using multiple miniature cross-axis inertial sensors (e.g., accelerometers) together with either collocated force actuators or piezoceramic actuators placed under each sensor. Collocated actuator/sensor pairs and decentralized (local) feedback may be optimized over the bandwidth of interest to ensure stability of multiple local feedback loops. For example, the control system may include an array of actuator/sensor pairs (e.g., n×n array of such actuator/sensor pairs, such as 4×4 or greater), which may be connected together with n² local feedback control loops. Using force actuators, significant frequency-averaged reductions up to 1 kHz in both the kinetic energy (e.g., 20-100 dB) and transmitted sound power (e.g., 10-60 dB) can be obtained with an appropriate feedback gain in each loop.

In certain variations, the signal processing system further comprises a second analog subsystem comprising a programmable gain amplifier configured to dynamically amplify at least the selected portion of the vibroacoustic signal, and an analog-to-digital converter providing a digitized biological vibroacoustic signal component.

1.s. Artificial Intelligence Module

Without the right algorithms to refine data, the real value of high-resolution sensor data obtained by embodiments of the present system 100 and method will remain hidden. Popular approaches such as neural nets model correlation, not causal relationships, and do not support extrapolation from the data. In contrast, Developers have developed a novel Structural Machine Learning (SML) platform, which is a natural feedforward and feedback platform, where data exploration and exploitation can be achieved faster and more accurately. Automatic expression synthesis tools build generalizable and evolving models, distilling the sensor data into human-interpretable form, yielding the true value of fused data in an intelligent, agile, networked, and autonomous sensing/exploitation system.

In this respect, some embodiments, the system 100 includes an artificial intelligence module which is configured to use machine learning and other forms of adaptation (e.g., Bayesian probabilistic adaptation) to optimize analytical software including data-driven feedback loops, for purposes of analyzing the vibroacoustic and/or other sensor data. The training of such machine learning models for analyzing data from the sensing device may begin with human-derived prior knowledge, or “soft knowledge” artefacts. These “soft knowledge” artefacts are advantageously generally much more expressible than off-the-shelf ML models like neural nets or decision trees. Furthermore, in contrast to mainstream machine learning scenarios that have clearly delineated training and test phases, analytical software for analyzing data from the sensing device may involve learning and optimizing software inline. In other words, the notion here is to embed an “inline learning” algorithm within an artificial intelligence (AI) software system, allowing the AI system to learn adaptively as the system processes new data. Such inline (and real-time) adaptation typically leads to more performant software AI systems with respect to various functional and nonfunctional properties or metrics, at least because (i) the AI system can correct for the suboptimal biases introduced by human designers and (ii) respond swiftly to changing characteristics operating conditions (mostly to variation in data being processed).

With respect to analyzing specifically vibroacoustic data, the vibroacoustic biofield harvested from patients may be saved as audio (.wav) files. Custom cross frequency coupling methodology, in combination with averaging wavelets such as Daubechies and Haar wavelet approaches, may be used to analyze the infrasound data as static images within set time windows. The Haar wavelet is the first and simplest orthonormal wavelet basis. Since the Daubechies wavelet averages over more data points, it is smoother than the Haar wavelet and may be more suitable for some applications. Typically, the audio scenes are of complex content, including background noise mixed with rich foreground having audible and inaudible vibrations and their context. In general, both background noise and foreground sounds can be used to characterize a “diagnostic scene” for use in characterizing a subject. Other data like contextual data could be converted into a visual 2D representation and attached to the static infrasound images to create a new image. Such new image is then analyzed as a whole to increase the performance of the algorithm.

However, foreground sounds typically occur in an arbitrary order, thereby making hidden sequential patterns hard to uncover. Thus, the ability to recognize and “unmask” a surrounding diagnosis environment by isolating and identifying contextualized audible and inaudible vibration signals has potential for many diagnostic applications. One approach to accomplish this is to shift from conventional classification techniques to modern deep neural networks (DNNs), and rand convolutional neural network (CNNs). However, despite their top performance, these network variants may not be sufficiently capable of modeling sequences in certain applications. Thus, in some variations the AI system may incorporate combined deep, symbolic, hybrid recurrent and convolutional neural network R/CNNs. Furthermore, in some variations, a separate DNN may generate and propose a “crisp” (symbolic) program, where feedback from execution of such a program may be used to tune/train the above DNNs and/or CNNs in a hybrid symbolic-subsymbolic approach.

In some variations, sensitivity of the sensing platform may be increased by using biophysiologically precise simulated patient entities for machine learning algorithm training purposes. For example, such simulated entities may be uploaded and modified in a training environment using high precision clinical data (e.g., heart rate, pulse rate, breathing rate, heart rate variability, breathing rate variability, pulse delay, core temperature, upper and lower respiratory temperature gradients, etc.) collected from well-characterized clinical patients to create a large, realistic training dataset.

In certain embodiments, the machine learning module is configured to (i) design a Covid-19 biosignature in a training phase using variations of the sensing devices and systems described herein, and/or (ii) apply the Covid-19 biosignature using variations of the sensing devices and systems described herein. In certain other embodiments, the machine learning module is configured determine unique biosignatures based on the sensor data and to apply the unique biosignatures to identify individual subjects, or groups of subjects.

Novel aspects of methods executed by the machine learning module comprise posing a machine learning problem as a task of program synthesis. To that aim, a domain-specific language (DSL) was designed to express various designs of a biosignature as programs in that language. In certain variations, inputs to the DSL comprise raw time series (detected frequency signals) as well as various types of features extracted from the series, like FFT spectrum, STFT spectrograms, MFCCs, vibe-scale features, peak locations, and more. These correspond to specific data types in the bespoke DSL. The DSL is equipped with functions (instructions) that can process inputs and variables of particular types. The DSL functions are based on domain specific knowledge. For instance, DSL functions we use now routinely include: convolution, peak finding, parameterizable low-pass and high-pass filters, arithmetic of time series, and more.

Importantly, these building blocks are defined on a much higher abstraction level than the typical vocabulary of SOTA ML techniques, where for instance deep learning models are essentially always nested compositions of dot products with nonlinearities. Secondly, they build upon the available body of knowledge that proved useful in signal processing and analysis in several past decades. Thirdly, the grammar of the DSL permits only operations that make sense in the context of signature identification, and can be used to convey experts' knowledge about the problem.

Expressing the models as programs can benefit from a wealth of theoretical and practical knowledge concerning the design and semantics of programming languages. Concerning data representation, we can rely on the formalized approach of type systems, which allow us to reason about data pieces, their relationship and their processing in a principled and sound way. To that aim, we rely on the fundamental formalism of algebraic data types, which allows systematic creation of new data types by aggregation and composition of existing types. In some variants (e.g. so-called dependent types), we can ‘propagate’ the properties of data through functions and so constrain their output types. Next, the actual processing of data can be conveniently phrased using recursion schemes, which provide a universal framework for aggregation and disaggregation of information for arbitrary, variable-size data structures (e.g. time series). Last but not least, the DSL is designed in a way that is compatible with the structure characteristic of a problem.

The above mechanisms can “regularize” the process of program synthesis and make it more likely to find a solution (program) that is plausible for a given problem, and in particular which does not overfit to the available training data, making valid generalization more likely. This makes it possible to synthesize robust signatures, classifiers and regression models from limited numbers of training examples.

### 2. Methods for Characterizing a Bodily Condition

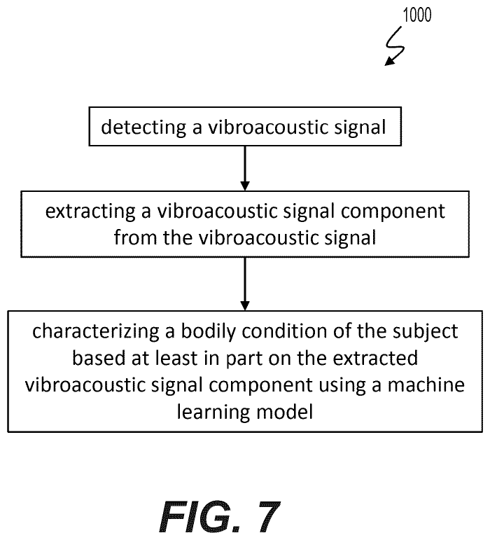

As shown in FIG. 7, in some variations, a method 1000 for characterizing a bodily condition may include detecting a vibroacoustic signal with the sensing device 110, extracting a vibroacoustic signal component from the vibroacoustic signal, and characterizing a bodily condition of the subject based at least in part on the extracted vibroacoustic signal component using, for example, a machine learning model. In some variations, instead of, or in addition to the vibroacoustic signal, the method 1000 may comprise obtaining data from any sensor described herein such as an optical sensor, a bioelectric sensor, a capacitive sensor, a thermal sensor, etc. The method may, at least in part, be executed by the processor 105 of the computer system 102. In certain embodiments, the bodily condition is COVID-19, and the method comprises detecting a vibroacoustic signal within a frequency range of: about 0.01 Hz to at least about 160 kHz. In certain other embodiments, the bodily condition is a unique identifier associated with the body, which can be used for identification or security purposes.

The sensor data may be obtained as a live stream. Alternatively, the sensor data may be sampled to provide sampled sensor data which is further processed. The data may be captured as catenated raw amplitude sequences or as combined short-time Fourier transform spectra. The data from the sensors may be captured from the subject in less than 15 seconds per subject, and preferably in less than 10 seconds per subject.

The method 1000 may comprise an optional prior step of causing the sensor 101 to start capturing the data based on a trigger. The trigger may be manual (e.g. initiated by a user of the system) or automatic and based on a predetermined trigger parameter. The trigger parameter may be associated with a proximity of the subject to the system 100, or a contact of a body part of the subject with the device, or on detection of a predetermined physiological parameter such as an elevated body temperature. The method 1000 may comprise causing the one or more sensors 101 to stop obtaining data based on a manual or automatic trigger. The automatic trigger may comprise a predetermined threshold such as a time interval or the like.

The processing of the data to determine a presence or absence of the bodily condition may take less than 15 seconds per subject, such as about 14 seconds, about 13 seconds, about 12 seconds, about 11 seconds, about 10 seconds, about 9 seconds, about 8 seconds, about 7 seconds, about 6 seconds, about 5 seconds, or less than 5 seconds. In certain variations, the vibroacoustic signal detected by the system spans between 3 and 5 heart beats of the subject.

Optionally, the method may comprise causing an output of the determination of the bodily condition to, for example, the device 109 described herein. The output may take any form such as an audio output (e.g. a beep), a visual output (e.g. a flashing light), a haptic output (e.g. a buzz), a mechanical output (e.g. barriers being opened or closed). In certain variations, the output may be an alert such as a green light indicating absence of the target condition or a red light indicating presence of the target condition. In other variations, the output may comprise causing the physical retention of the subject through control of a physical restraint member such as a barrier.

In some embodiments, transmitted sensor data may be encrypted before being saved to personalized folders for secure storage and subsequent playback in a mobile application executed by a mobile computing device. The application may provide the ability to save collected data within designated Electronic Medical Records (EMR)/Electronic Health Record (EHR) systems, share patient recordings, and annotate notes on recorded audio, etc. (Data pre-processing)

One or more of the sensor data, the determination of the bodily condition and the output may be stored, such as in a database of the computing system 102. The stored data may be fed to a training MLA.

The processing the sensor data or training the MLA comprises associating a given target condition with symptoms of the given target condition. The symptoms may include one or more symptoms related to the subject's throat, chest, constitution, gut, nasal system, eyes, and vascularization. These symptoms may include, but are not limited to a sore, painful, swollen, or scratchy throat, loss of taste, or difficulty in swallowing. The chest associated symptoms are trouble breathing, congestion, tightness, dry cough, hacking cough, wet cough, loose cough, mucous, phlegm or fibrosis. The constitutional symptoms may be dyspnea, muscle spasms, pyrexia, body aches, fatigue, malaise, general discomfort, fever, or chills. The gut associated symptoms may be loss of appetite, altered gut motility, stomachache, emesis, nausea, or diarrhea. The nasal symptoms may be rhinorrhea, redness of the nasal openings or congestion. The ocular symptoms may be glassy eyes and conjunctival injection. The vascularization symptoms may include clotting, bruising, etc. For example, sore throat, dry cough, shortness of breath, muscle spasm, chills, fever, gut discomfort, brain fog and diarrhea are key indicators of a possible coronaviridae (e.g. COVID-19) infection.

These and other indicative symptoms that can be detected in a non-invasive, contactless manner by variants of the present technology are typically due to changes in tracheal and lung thickness, respiratory depression, local and systemic fluid accumulation (edema), oxygen desaturation, hypercapnia, trauma, scarring, tissue irritation, fibrotic changes, hypoventilation and hypertension. Variants of the present technology can be used to detect early and subtle changes in lung and upper respiratory airway audible and inaudible wheezes, crackles, and egophony—often caused by lung consolidation, diffuse alveolar damage, vascular injury, and/or fibrosis, with or without ECG.

Other physiological states or levels of metabolites or environmental toxins that can be detected by the method include mechanical trauma and injury, elevated interleukin (IL) 6 and polymorphonuclear inflammatory cells and mediators, lymphoid hypertrophy and prominence of adenoidal and tonsillar tissue, kinins, histamine, leukotrienes, prostaglandin D2, and TAME-esterase, ACE inhibitor increase in pro-inflammatory pharyngeal irritation, oropharyngeal mucositis, and the direct effect of ozone on respiratory tract cell membranes and fluid, lipid ozonation product activation of specific lipases that trigger the release of endogenous mediators of inflammation such as prostaglandin E, IL8, thromboxane B2 and calcitonin gene-related peptide.

In some variations, the method may be performed with any of the systems 100 or sensing devices 110 described herein, which may have any suitable variation of the sensing device 110 or sensor 101 and/or other sensors.

In some variations, the extracted vibroacoustic signal component may include a biological vibroacoustic signal component, and the bodily condition characterized may include a health condition based on the biological vibroacoustic signal component. In this respect, the method may comprise extracting the biological vibroacoustic signal component. In certain variations, extracting the biological vibroacoustic signal component comprises passing the vibroacoustic signal through a first stage amplifier and a first stage low pass filter, and a second stage amplifier and a second stage low pass filter. The first stage low pass filter and the second stage low pass filter may form a second order low pass filter with anti-aliasing, wherein the second order low pass filter has a cutoff frequency of about 15 kHz to about 20 kHz. In certain variations, the vibroacoustic signal is also passed through a third stage amplifier comprising a programmable gain amplifier configured to dynamically amplify at least a portion of the vibroacoustic signal. In certain variations, the extracting the biological vibroacoustic signal component comprises digitizing the amplified portion of the vibroacoustic signal and providing at least a portion of the digitized vibroacoustic signal as a digitized biological vibroacoustic signal component.

For example, the method 1000 may assist healthcare professionals in collecting and intelligent analysis of audible and inaudible signals associated with cardiac, lung, gut and other internal organ functions, for rapid and accurate diagnostics such as that relating to cardiopulmonary, respiratory, and/or gastrointestinal function. In certain variations, the method may assist in the diagnosis of a viral infection, such as that of a Covid-19 or SARS virus. In certain variations, the method may assist in monitoring efficacy of a certain treatment, such as during a clinical trial.

The method 1000 may include collecting data generated by the body passively without imparting any energy (e.g., current or voltage) to the body.

The sensing devices, sensors, systems, and methods of the current technology may be useful in detecting bodily conditions in living organisms including but not limited to: respiratory illnesses and diseases such as COVID-19, SARS, digestive illnesses and diseases, cancer, Neurological illnesses and diseases, psychiatric illnesses and diseases, cardiac illnesses and diseases, circulatory illnesses and diseases, lymphatic illnesses and diseases, kidney illnesses and diseases, liver illnesses and diseases, lung illnesses and diseases, osteopathic illnesses and diseases, orthopedic illnesses and diseases, sleep related illnesses and diseases, metabolic diseases, disorders, and states, movement disorders, viral, bacterial, fungal, parasitic, protozoal, and prion infections, substance use disorders, behavioral disorders, musculoskeletal illnesses and diseases, blood illnesses and diseases, disfunction of internal organs, genital illnesses and diseases, emotional disturbances, disorders or states, alertness, fatigue, anxiety, depression, delirium, disorientation, ataxia, insomnia, eating disorders, obesity, body composition, and such.

In certain aspects, the bodily condition determination is subject to type I errors less than 0.1%, 0.2%, 0.3%, 0.4%, 0.5%, 0.6%, 0.7%, 0.8%, 0.9% or 1%. In certain aspects, the bodily condition determination is subject to type II errors less than 0.1%, 0.2%, 0.3%, 0.4%, 0.5%, 0.6%, 0.7%, 0.8%, 0.9% or 1%. In certain aspects, bodily condition determination is subject to type I errors less than 1.1%, 1.2%, 1.3%, 1.4%, 1.5%, 1.6%, 1.7%, 1.8%, 1.9% or 2%. In certain aspects, the bodily condition determination is subject to type II errors less than 1.1%, 1.2%, 1.3%, 1.4%, 1.5%, 1.6%, 1.7%, 1.8%, 1.9% or 2%. In certain aspects, the bodily condition determination is subject to type I errors less than 2.1%, 2.2%, 2.3%, 2.4%, 2.5%, 2.6%, 2.7%, 2.8%, 2.9% or 3%. In certain aspects, the bodily condition determination is subject to type II errors less than 2.1%, 2.2%, 2.3%, 2.4%, 2.5%, 2.6%, 2.7%, 2.8%, 2.9% or 3%. In certain aspects, the bodily condition determination is subject to type I errors less than 3.1%, 3.2%, 3.3%, 3.4%, 3.5%, 3.6%, 3.7%, 3.8%, 3.9% or 4%. In certain aspects, the bodily condition determination is subject to type II errors less than 3.1%, 3.2%, 3.3%, 3.4%, 3.5%, 3.6%, 3.7%, 3.8%, 3.9% or 4%. In certain aspects, the bodily condition determination is subject to type I errors less than 4.1%, 4.2%, 4.3%, 4.4%, 4.5%, 4.6%, 4.7%, 4.8%, 4.9% or 5%. In certain aspects, the bodily condition determination is subject to type II errors less than 4.1%, 4.2%, 4.3%, 4.4%, 4.5%, 4.6%, 4.7%, 4.8%, 4.9% or 5%. In certain aspects, the bodily condition determination is subject to type I errors less than 6%, 7%, 8%, 9%, 10%, 15%, 16%, 17%, 18% or 19%. In certain aspects, the bodily condition determination is subject to type II errors less than 6%, 7%, 8%, 9%, 10%, 15%, 16%, 17%, 18% or 19%.

In certain aspects, above levels of accuracy are achieved with less than 2, 3, 4, 5, 6, or 7 sensors 101. In certain aspects, a throughput of the system 100 ranges from at least one hundred subjects scanned per hour to about one thousand subjects scanned per hour. In certain embodiments, the throughput is about 500 subjects scanned per hour. In some variations, the method for characterizing a bodily condition may include detecting vibroacoustic signals with active skin motion amplification methods in the sensing device 110.

### EXAMPLES

### Example 1—Design of Voice Coil Sensor

Developers goal was to develop a novel voice coil-based sensor for human/animal physical monitoring. Among the optimization parameters were:
magnet size, shape and material for strong and uniform magnetic field;
coil material, length, number of windings and number of layers for maximum length while minimizing resistance and weight;
lightweight support structure e.g. flexure-based structure, or flexible diaphragm holding windings;
overall dimensions constrained because of the need to incorporate the sensor into lightweight small wearable device or handheld device.

Performance of candidate transducer designs was evaluated based on three output variables: force responsivity, receiving sensitivity, and frequency-response efficiency.

### Force Responsivity

The actuating abilities of the transducers were evaluated by measuring the force exerted by the transducers as a function of frequency. A Dynamic Signal Analyzer (DSA) was used to step through source frequencies in the range of 0.01 to 160,000 Hz and to calculate a frequency response spectrum of the signal from the force transducer measured for each source frequency.

### Receiving Sensitivity

The DSA was used to step through a range of source frequencies and to calculate a frequency response spectrum of the receive signal from the test transducer, measured at each source frequency.

### Reciprocal Concrete Transmission Efficiency

To verify that it is indeed possible to collect and transmit infrasound through to ultrasound mechano-acoustic waves passively harvested from the human body using these test voice coil transducers, measurements were made from patients and on a physiologic manikin Two identical transducers were used for this experiment: one for transmission and one for reception. The reciprocity in the transmissions was investigated by repeating each measurement with reversed transducer configuration, so that the transducer that previously transmitted acted as receiver and vice versa.

The DSA source was used, via the power amplifier, to apply a sinusoidal signal with stepped frequency to the transducer that acted as a transmitter. The output of the power amplifier was fed into Ch. 1 of the DSA for reference. The output from the receiving transducer was fed into the DSA (Ch. 2). By dividing Ch. 2 with Ch. 1 the voltage transfer function was established. By then dividing by the transducer complex impedance the transmission efficiency was determined.

TABLE 1
Voice coil parameter ranges in certain variants of the present technology.

| Parameter | Present technology 1 | | Present technology 2 | | Conventional voice coil | |
|---|---|---|---|---|---|---|
| Impedance | 150 ohms ±2% | | 150 ohms ±2% | | 4 | ohms |
| DC Resistance (Re) | 150 ohms ±2% | | 150 ohms ±2% | | 4.3 | ohms |
| Voice Coil Inductance (Le) | 7.5 mH at 1 kHz/ | | 8.46 mH at 1 kHz/ | | 0.27 mH at 1 kHz/ | |
| | 2.5 mH at 10 kHz | | 2.7 mH at 10 kHz | | 0.12 mH at 10 kHz | |
| Coil Resonant Frequency | 80-170 | Hz | 90 Hz ±2% | | 224 | Hz |
| (Fs) | | | | | | |
| Total Q (Qts) Inverse of | 0.25 to 0.65 | | 0.85-0.90 | | 0.78 | |
| damping | (depending on exciter) | | | | | |
| | 100 mg to 100 g | | | | | |
| | depending on no. | | | | | |
| | windings) | | | | | |
| Moving Mass (Mms) | 100 mg to 100 g | | 1.15 | g | 1.61 | g |
| | (depends on number | | | | | |
| | of windings) | | | | | |
| | For test exciters | | | | | |
| | specifically 1.15 g | | | | | |
| Mechanical Compliance of | 0.4 to 3.2 mm/N | | 3.2 | mm/N | 0.338 | mm/N |
| Suspension (Cms) (inverse | | | | | | |
| of suspension) | | | | | | |
| BL Product (BL) | 18.5 N/Amp (same | | 18.5 | Tm | 3.63 | Tm |
| | as Tm) | | | | | |
| Voice Coil Diameter | 25 | mm | 25 | mm | 25 | mm |
| RMS Power Handling | 2 | W | 2 | W | 24 | watts |

| Wire Diameter | 0.05 | mm | 0.05 | mm | 0.15 (including |
|---|---|---|---|---|---|
| | | | | | insulation) |
| Number of windings | 208 | | 208 | | 46 |
| Number of Layers | 4 | | 4 | | 2 |
| Magnet Size | 24 mm × 3.5 mm | | 24 mm × 3.5 mm | | 24 mm × 3.5 mm |
| Overall Outside Diameter | 50.5 mm (5 × 5 × 0.1 to | | 60 mm and 65 mm (oval | | 50.5 |
| | 50 × 50 × 10) | | shaped) | | |
| Overall Depth | 20.5 | mm | 27 | mm | 20.5 |

| | | | |
|---|---|---|---|
| Inductance/moving mass | at least 6.52 mH per | 7.36 mH per | 10.17 mH per |
| ratio | gram at 1 kHz | gram at kHz | gram at 1 kHz |
| Mechanical compliance/ | at least 0.348 mm/N | 2.78 mm/N per gram | 0.21 mm/N per gram |
| moving mass ratio | per gram | | |
| BL product/moving mass | at least 16 N/Amp | 16.09 N/Amp per gram | 2.25 N/Amp per gram |
| ratio | per gram | | |
| (BL × mechanical | 51.48 [T*m\hat{ }2/(N * g)] | 51.48 [T*m\hat{ }2/(N * g)] | 0.76 [T*m\hat{ }2/(N * g)] |
| compliance)/moving mass | | | |
| Wright Parameters | | | |
| K(r) | 26-27 | 23 | 0.275 |
| X(r) | 0.175-0.185 | 0.194 | 0.286 |
| K(i) | 0.00709-0.01118 | 0.032 | 0.00045 |
| X(i) | 0.827-0.866 | 0.739 | 0.843 |

Parameters that may lead to high sensitivity and frequency range (higher=better): Voice Coil inductance, Total Q, mechanical compliance, BL product, number of windings (resulting in higher BL and Inductance). Parameters that may lead to high sensitivity and frequency range (lower=better): Moving mass.

The product of BL product and mechanical compliance may represent high signal sensitivity amplified by good mechanical compliance.

The product of BL product and mechanical compliance)/mass may represent high signal sensitivity amplified by good mechanical compliance, further amplified by low moving mass.

By way of background, and to support the abovedescribed experimental approach, the following was considered. An electrodynamic sensoriactuator is a reversible voice coil transducer which has capability to provide input vibrational energy to a host mechanical structure. It can be regarded as a two-port system, including electromechanical coupling through two pairs of dual variables: the voltage e and current i for the electrical side, and the transverse force Fₛ and velocity vₛ for the mechanical side.

Using phasors to represent the complex amplitude (magnitude and phase) of sinusoidal functions of time, the characteristic equations of the sensoriactuator when attached to a host mechanical structure can be written as:

Bli=Zₘₐvₐ−Zₘₛvₛ (1)

e=Zₑi−ε (2)

where vₐ is the velocity of the moving mass, vₛ, is the transverse velocity at the base of the actuator, e is the input voltage applied to the electrical terminals, i is the current circulating in the coil, Zₘₐ=jωMₐ+Rₐ+Kₐ/jω is the mechanical impedance of the inertial exciter, Zₑ=Rₑ+jωLₑ is the blocked electrical impedance of the transducer, and Zₘₛ=Rₐ+Kₐ/jω is the impedance of the spring-dashpot mounting system. Equations 7-8) contain terms of electrodynamic coupling; F_{mag}=Bli is the force caused by the interaction of the magnetic field and the moving free charges (current), and ε=Bl(vₐ vₛ) is the back electromotive force (voltage) induced within the voice coil during motion. It is also assumed that all the forces acting on the actuator are small enough so that the displacements remain proportional to applied forces (small-signal assumptions).

The input impedance of the sensoriactuator is the complex ratio of the voltage to the current in the electrical circuit of the transducer. It determines the electrical impedance (in Ω) “seen” by any equipment such as electronic drive source, electrical network, etc., connected across its input terminals. When attached to a pure mass, the closed form expression of the input impedance of the sensoriactuator can be obtained by combining Eq. (1) and (2), as

$$Zin = \frac{e}{i} = {Z}_{e} + \frac{{( Bl )}^{2}}{{Z}_{ma}} \qquad ( 3 )$$

As can be seen in Eq. (3), Zᵢₙ contains all the electromechanical effects that are operating, including all resistances and reactances of the actuator impedance. As discussed in the following, measuring the input impedance of the actuator enables certain key parameters such as the dc resistance and natural frequency to be evaluated.

Substituting now Eq. (1) in Eq. (2), the transverse velocity at the base of the actuator be expressed as:

$$γ = \frac{Zma}{jwMaBl} ( e\_Zei ) + \frac{Bl}{jwMa} i \qquad ( 4 )$$

Equation (4) clearly shows that the transverse velocity of the structure where the actuator is located can be estimated from the driving current and the voltage sensed at its input terminals.

### Example 2—Remote Sensing

An example variation of the sensing device 110 of the present technology. The sensing device had a sealed cavity. A subject was positioned at varying distances from the diaphragm 116 of the sensing device 110 and including different barriers between the subject and the diaphragm in terms of apparel (wearing a sweater, without a sweater).

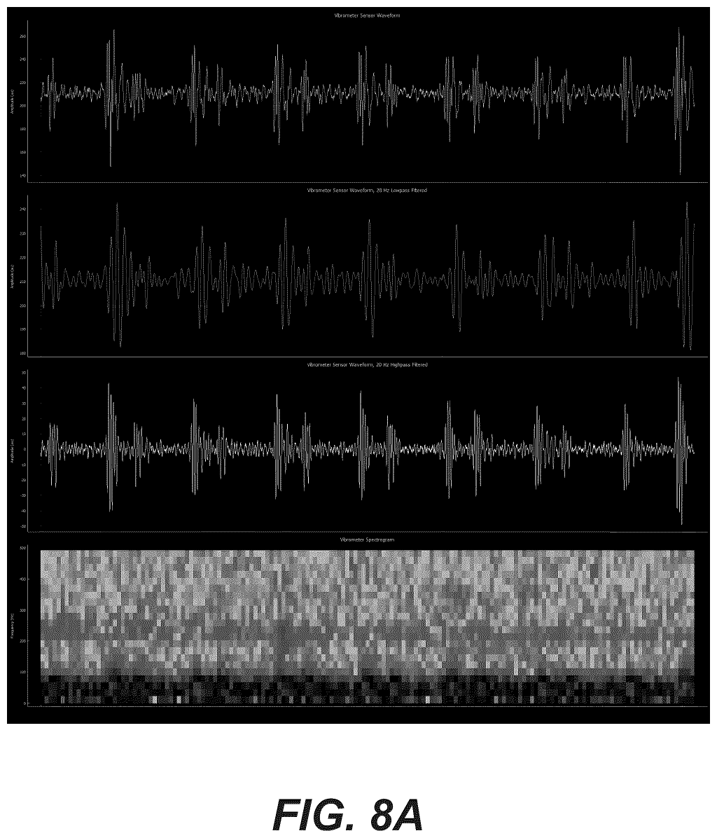

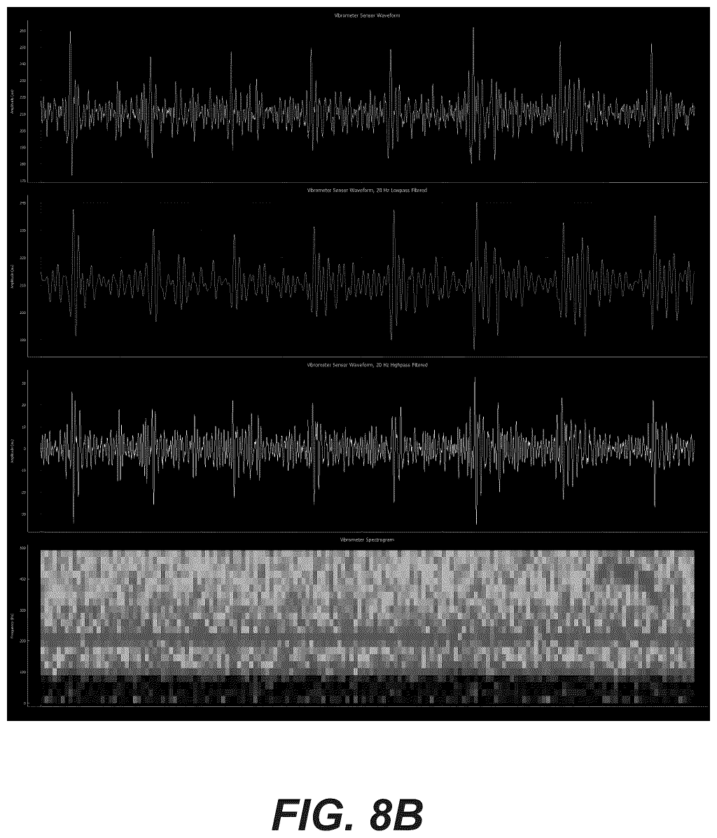

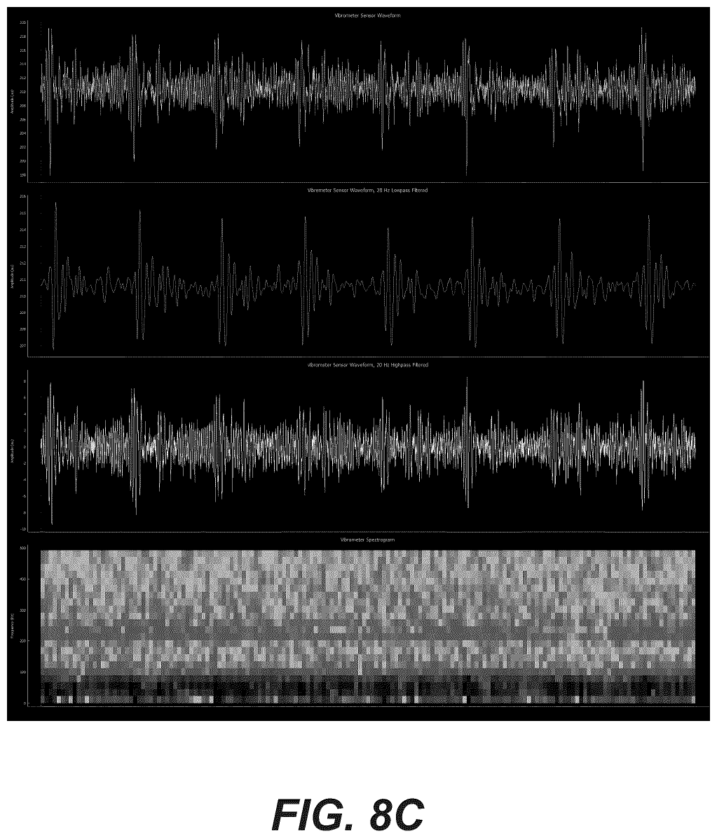

FIG. 8 shows vibroacoustic test data collected by the sensing device when the subject without a clothing barrier is positioned 12 cm from the diaphragm of the sensing device (FIG. 8A), the subject without a clothing barrier is positioned 100 cm from the diaphragm of the sensing device 100 cm away (FIG. 8B), the subject wearing a sweater is positioned 12 cm from the diaphragm of the sensing device (FIG. 8C), and the subject wearing a sweater is positioned 100 cm from the diaphragm of the sensing device (FIG. 8D).

It is clear from the figures that systems and sensing devices of the present technology can detect body vibrations remotely through air gaps of various distances. Due to attenuation inherently present in the propagating sound signals the amplitude is reduced at greater distances, as most evident in the time domain signals. However, as evident from the frequency spectra, signals are still being captured at the larger distance and can be extracted. In addition, the important lower frequencies are less attenuated due to near field conditions, which allows sensing them from even greater distances. As can also be seen, the presence of clothing has little effect on the signal quality.

FIG. 8 shows vibroacoustic test data collected by the sensing device when the subject wearing a sweater is positioned 10 cm from a diaphragm of the sensing device and is facing the diaphragm (FIG. 8A), the subject wearing a sweater is positioned 10 cm from a diaphragm of the sensing device and is facing away from the diaphragm (FIG. 8B), and the subject wearing a sweater is positioned 100 cm from a diaphragm of the sensing device and is facing the diaphragm. These signals are presented as time domain signals, and separated into relevant frequency bandwidths. The top cyan colored signal is the combined captured signal, the green signal the infrasound component in the captured signal (<20 Hz), the yellow signal is the audible component (>20 Hz) and the bottom is a spectrogram. It is evident that infrasound and audible spectrum are captured with good signal to noise ratio. Although frequencies in the audible spectrum are attenuated compared to a distance of 10 cm, the infrasound components are still captured well.

It is reasonably expected that when openings are provided on the back cover, the effect on frequency detected would be affected (i.e. shifted to higher frequencies). Optimization of the sensing device can therefore be performed through a combination of analytical and finite element analysis (FEM) of different extents of sealing of the cavity and based on a desired frequency detection range in a high dimensional parameter space.

The foregoing description, for purposes of explanation, used specific nomenclature to provide a thorough understanding of the invention. However, it will be apparent to one skilled in the art that specific details are not required in order to practice the invention. Thus, the foregoing descriptions of specific variations of the invention are presented for purposes of illustration and description. They are not intended to be exhaustive or to limit the invention to the precise forms disclosed; obviously, many modifications and variations are possible in view of the above teachings. The variations were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

1. A system for non-contact monitoring of acoustic signals associated with a body, the system comprising:
a sensing device comprising:
a support member defining an aperture,
a diaphragm extending across the aperture such that at least a portion of the diaphragm covers the aperture, and
a sensor connected to the support member or the membrane and configured to convert movement of the diaphragm to electric signal data.

2. The system of claim 1, wherein the sensor is configured to detect acoustic signals having a frequency ranging from about 0.01 Hz to at least about 160 kHz.

3. The system of claim 1, further comprising a computing system, including a processor, communicatively coupled to the sensing device and configured to execute a method for determining a bodily condition of the body based on the electric signal data.

4. The system of claim 2, wherein the processor is configured to filter the electric signal data to remove electric data not associated with the body, the determining the bodily condition being based on the filtered electric signal data.

5. The system of claim 4, wherein the body is a human or animal subject, and the filtering the electric signal data comprises the processor removing electric signal data which is not associated with a physiological parameter of the human or animal subject.

6. The system of claim 3, wherein the method for determining a bodily condition based on the electric signal comprises executing a trained machine learning algorithm.

7. The system of any of claims 1-6, wherein the support member is a frame having a first side and a second side and the aperture extends through the frame between the first side and the second side, wherein the diaphragm covers the aperture on one of the first side and the second side.

8. The system of claim 7, further comprising a back cover to cover the aperture on the other of the first side and the second side.

9. The system of claim 7, wherein the diaphragm is configured to seal the aperture.

10. The system of any of claims 1-6, wherein the support member comprises a frame having a first side and a second side, wherein the aperture is formed in one of the first side and the second side and does not extend therethrough.

11. The system of any of claims 1-6, wherein the sensor comprises:
a voice coil component comprising a coil holder supporting wire windings;
a magnet component comprising a magnet supported by a magnet housing, the magnet having a magnet gap configured to receive at least a portion of the voice coil component in a spaced and moveable manner;
a connector connecting the voice coil component to the magnet component, the connector being compliant and permitting relative movement of the voice coil component; wherein one of the voice coil component and the magnet component is connected to the diaphragm such that movement of the diaphragm induces a relative movement between the voice coil component and the magnet component.

12. The system of claim 11, wherein the diaphragm is attached to the voice coil component and the wire windings are spaced from the diaphragm.

13. The system of any of claims 1-6 wherein the sensor comprises an electric potential sensor which is attached to the support member and spaced from the diaphragm.

14. The system of claim 13, wherein the electric potential sensor is positioned in a cavity of the aperture, or outside of the cavity.

15. The system of claim 13, further comprising a conductive layer on the diaphragm.

16. The system of any of claims 1-6, whereon the sensor is one or more selected from: a voice-coil type sensor, an electric potential sensor, a capacitive sensor, a magnetic field disturbance sensor, a photodetector and light source, a strain sensor, an Inertial Measurement Unit (IMU), and an acoustic echo doppler.

17. The system of any of claims 1-6, further comprising a plurality of sensors arranged as an array relative to the support member.

18. The system of claim 17, wherein each sensor of the plurality of sensors is supported by a respective support member.

19. The system of claim 17, wherein each sensor of the plurality of sensors is configured to detect a different frequency range of acoustic signals.

20. The system of claim 17, wherein the diaphragm is connected to each support member to close or fluidly seal a respective aperture.

21. The system of claim 17, wherein the diaphragm is connected to an outer mount which contains the support members of the plurality of sensors.

22. The system of any of claims 1-6, wherein the sensing device further comprises a front cover connected to the support member and covering the diaphragm.

23. The system of any of claims 1-6, wherein the sensor is positioned relative to the diaphragm by one or more supports extending from the frame.

24. The system of claim 2, further comprising at least one additional sensor communicatively coupled to the processor.

25. The system of claim 24, wherein the at least one additional sensor is selected from a heat sensor, a humidity sensor, a barometric pressure sensor, an ambient noise sensor, an ambient light sensor, an ultrasound sensor, an altitude sensor, a camera, a volatile organic compound sensor, ACG, BCG, ECG, EMG, EOG, SCG, and UTI.

26. A method for non-contact monitoring of acoustic signals associated with a body, the method executed by a processor of a system defined in claim 1, the method comprising:
obtaining vibroacoustic data detected by the sensing device of claim 1 operatively communicable with the processor;
extracting, from the detected vibroacoustic signal, a vibroacoustic signal component originating from the subject; and
characterizing presence or absence of a bodily condition of the body based at least in part on the extracted vibroacoustic signal component.
